(19) United States
(12) Patent Application Publication
(10) Pub. No.: US 2018/0105478 A1
(43) Pub. Date: Apr. 19, 2018

(54) Process for Reducing the Light Oligomer Content of Polypropylene Oils

(71) Applicant: Chevron Phillips Chemical Company LP, The Woodlands, TX (US)

(72) Inventors: Graham R. Lief, Bartlesville, OK (US); Uriah J. Kilgore, Kingwood, TX (US); Eric J. Haschke, Bartlesville, OK (US)

(21) Appl. No.: 15/297,179

(22) Filed: Oct. 19, 2016

(51) Int. Cl. C07C 2/32; B01J 31/18; B01J 31/14; C08F 10/00

(52) U.S. Cl. CPC C07C 2/32; B01J 31/1835; B01J 31/143; B01J 2531/847; C07C 2531/18; B01J 2231/20; B01J 2531/824; C08F 10/00

(57) ABSTRACT

Disclosed herein are catalyst systems containing an alpha-diimine nickel halide complex, a chemically-treated solid oxide, and an optional co-catalyst. These catalyst systems can be used to reduce the light oligomer content of propylene oligomer streams, for instance, by oligomerizing olefin feedstocks containing C₆ to C₂₇ propylene oligomers to produce oligomer compositions having higher molecular weights.

### BACKGROUND OF THE INVENTION

Many commercial propylene oligomerization processes result in oligomer products containing C₃₀+ (C₃₀ and above) propylene oligomers. However, a significant portion of the oligomer product can be a light oligomer fraction, typically encompassing C₆-C₂₇ propylene oligomers. This light oligomer fraction increases the volatility and flash point of the oligomer product, and often is removed by distillation and other suitable techniques.

It would be beneficial, however, to upgrade the light oligomer fraction to higher molecular weights, such that it could be combined with the C₃₀+ propylene oligomer fraction, thereby reducing waste, eliminating additional separations processes, and increasing the overall yield of C₃₀ propylene oligomers. Accordingly, it is to these ends that the present invention is generally directed.

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Aspects of this invention are directed to a process comprising contacting an olefin feedstock comprising C₆ to C₂₇ propylene oligomers with a catalyst composition comprising (i) an alpha-diimine nickel halide complex, (ii) a chemically-treated solid oxide, and (iii) optionally, a co-catalyst, to produce an oligomer composition. Beneficially, the process can result in an increase in the number-average molecular weight (Mn) of the oligomer composition as compared to that of the olefin feedstock. Moreover, in particular aspects of this invention, the ratio of higher oligomers to lower oligomers (C₁₈+/C₁₅−) of the oligomer composition can be greater than that of the olefin feedstock by at least 25% or 100%, and often up to 1000% or more. Therefore, the processes disclosed herein can be used to reduce the light oligomer fraction of an olefin stream, such that waste is reduced and the overall yield to C₃₀+ propylene oligomers can be increased.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and/or feature disclosed herein, all combinations that do not detrimentally affect the designs, compositions, processes, and/or methods described herein are contemplated with or without explicit description of the particular combination. Additionally, unless explicitly recited otherwise, any aspect and/or feature disclosed herein can be combined to describe inventive features consistent with the present disclosure.

While compositions and methods are described herein in terms of “comprising” various components or steps, the compositions and methods can also “consist essentially of” or “consist of” the various components or steps, unless stated otherwise. For example, a catalyst composition consistent with aspects of the present invention can comprise; alternatively, can consist essentially of; or alternatively, can consist of, an alpha-diimine nickel halide complex, a chemically-treated solid oxide, and a co-catalyst.

The terms “a,” “an,” “the,” etc., are intended to include plural alternatives, e.g., at least one, unless otherwise specified. For instance, the disclosure of “a chemically-treated solid oxide” or “a co-catalyst” is meant to encompass one, or mixtures or combinations of more than one, chemically-treated solid oxide or co-catalyst, respectively, unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

For any particular compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and one carbon-carbon double bond; a general reference to pentane includes n-pentane, 2-methyl-butane, and 2,2-dimethylpropane; a general reference to a butyl group includes an n-butyl group, a sec-butyl group, an iso-butyl group, and a t-butyl group; a general reference to cyclododecatriene includes all isomeric forms (e.g., trans,trans,cis-1,5,9-cyclododecatriene, and trans,trans,trans-1,5,9-cyclododecatriene, among other dodecatrienes); and a general reference to 2,3-pentanediol includes 2R,3R-pentanediol, 2S,3S-pentanediol, 2R,3S-pentanediol, and mixtures thereof.

The terms “catalyst composition,” “catalyst mixture,” “catalyst system,” and the like, do not depend upon the actual product or composition resulting from the contact or reaction of the initial components of the disclosed or claimed catalyst composition/mixture/system, the nature of the active catalytic site, or the fate of the co-catalyst, the nickel halide complex, or the chemically-treated solid oxide, after combining these components. Therefore, the terms “catalyst composition,” “catalyst mixture,” “catalyst system,” and the like, encompass the initial starting components of the composition, as well as whatever product(s) may result from contacting these initial starting components, and this is inclusive of both heterogeneous and homogenous catalyst systems or compositions. The terms “catalyst composition,” “catalyst mixture,” “catalyst system,” and the like, can be used interchangeably throughout this disclosure.

The terms “contact product,” “contacting,” and the like, are used herein to describe compositions and methods wherein the components are contacted together in any order, in any manner, and for any length of time, unless otherwise specified. For example, the components can be contacted by blending or mixing. Further, unless otherwise specified, the contacting of any component can occur in the presence or absence of any other component of the compositions and methods described herein. Combining additional materials or components can be done by any suitable method. Further, the term “contact product” includes mixtures, blends, solutions, slurries, reaction products, and the like, or combinations thereof. Although “contact product” can, and often does, include reaction products, it is not required for the respective components to react with one another. Similarly, the term “contacting” is used herein to refer to materials which can be blended, mixed, slurried, dissolved, reacted, treated, or otherwise contacted in some other manner. Hence, “contacting” two or more components can result in a mixture, a reaction product, a reaction mixture, etc.

The term “hydrocarbon” whenever used in this specification and claims refers to a compound containing only carbon and hydrogen. The term “olefin” as used herein refers to a hydrocarbon that has at least one carbon-carbon double bond that is not part of an aromatic ring or ring system. The term “olefin” includes aliphatic and aromatic, cyclic and acyclic, and/or linear and branched compounds having at least one carbon-carbon double bond that is not part of an aromatic ring or ring system, unless specifically stated otherwise. Olefins having only one, only two, only three, etc., carbon-carbon double bonds can be identified by use of the term “mono,” “di,” “tri,” etc., within the name of the olefin. The olefins can be further identified by the position of the carbon-carbon double bond(s).

The terms “oligomer composition” and “oligomer product” include all products made by the “oligomerization” process including the “oligomers” and products which are not “oligomers” (e.g., polymer). As used herein, “heavy propylene oligomer” typically refers to a propylene oligomer (or composition) having little to no light propylene oligomers, e.g., where at least a portion of lighter oligomers (such as C₆ to C₂₇ oligomers) has been removed. This term also can be used generically herein to include propylene homo-oligomers, propylene co-oligomers, and so forth.

Although any methods and materials similar or equivalent to those described herein can be used in the practice or testing of the invention, the typical methods and materials are herein described.

All publications and patents mentioned herein are incorporated herein by reference for the purpose of describing and disclosing, for example, the constructs and methodologies that are described in the publications, which might be used in connection with the presently described invention.

### DETAILED DESCRIPTION OF THE INVENTION

The present invention is directed generally to oligomerization catalyst compositions, methods for using the catalyst compositions to oligomerize propylene oligomer feedstocks, the oligomer compositions produced using such catalyst compositions, and formulations and other products produced using these oligomer compositions. In particular, the present invention relates to processes that comprise contacting an olefin feedstock comprising C₆ to C₂₇ propylene oligomers with a catalyst composition comprising an alpha-diimine nickel halide complex, a chemically-treated solid oxide, and an optional co-catalyst, to produce an oligomer composition. Beneficially, these processes can result in a reduction in the light oligomer content of the olefin feedstock, typically quantified by an increase in the Mn of the oligomer composition as compared to that of the olefin feedstock, or by an increase in the ratio of higher oligomers to lower oligomers (C₁₈+/C₁₅−) of the oligomer composition as compared to that of the olefin feedstock.

### Nickel Halide Complexes

Catalyst compositions in accordance with this invention can comprise an alpha-diimine nickel halide complex. In one aspect, for instance, the catalyst composition can comprise an alpha-diimine nickel chloride complex, while in another aspect, the catalyst composition can comprise an alpha-diimine nickel bromide complex.

The alpha-diimine nickel halide complex, in particular aspects of this invention, can have the following formula:

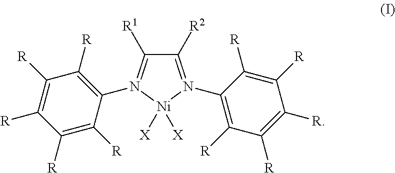

Within formula (I), R¹, R², each R, and each X are independent elements of the alpha-diimine nickel halide complex. Accordingly, the alpha-diimine nickel halide complex having formula (I) can be described using any combination of R¹, R², R, and X disclosed herein.

Unless otherwise specified, formula (I) above, any other structural formulas disclosed herein, and any nickel halide complex, compound, or species disclosed herein are not designed to show stereochemistry or isomeric positioning of the different moieties (e.g., these formulas are not intended to display cis or trans isomers, or R or S diastereoisomers), although such complexes or compounds are contemplated and encompassed by these formulas and/or structures.

Each R in formula (I) independently can be any suitable substituent. In some aspects, suitable substituents can include, but are not limited to, H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group. It is contemplated that each R can be either the same or a different substituent group. In one aspect, each R independently can be H, a halide (e.g., F, Cl, Br, etc.), a C₁ to C₁₈ hydrocarbyl group, a C₁ to C₁₈ halogenated hydrocarbyl group, a C₁ to C₁₈ hydrocarboxy group, or a C₁ to C₁₈ hydrocarbylsilyl group. In another aspect, each R independently can be H, a halide, a C₁ to C₁₂ hydrocarbyl group, a C₁ to C₁₂ halogenated hydrocarbyl group, a C₁ to C₁₂ hydrocarboxy group, or a C₁ to C₁₂ hydrocarbylsilyl group. In yet another aspect, each R independently can be H, a halide, a C₁ to C₈ hydrocarbyl group, a C₁ to C₈ halogenated hydrocarbyl group, a C₁ to C₈ hydrocarboxy group, or a C₁ to C₈ hydrocarbylsilyl group. In still another aspect, each R independently can be H, a halide, or a C₁ to C₁₈ hydrocarbyl group; alternatively, H or a C₁ to C₁₈ hydrocarbyl group; alternatively, H, a halide, or a C₁to C₈ alkyl group; or alternatively, H or a C₁ to C₈ alkyl group.

The hydrocarbyl group which can be an R in formula (I) can be a C₁ to C₃₆ hydrocarbyl group, including, but not limited to, a C₁ to C₃₆ alkyl group, a C₂ to C₃₆ alkenyl group, a C₄ to C₃₆ cycloalkyl group, a C₆ to C₃₆ aryl group, or a C₇ to C₃₆ aralkyl group. For instance, each R independently can be a C₁ to C₁₈ alkyl group, a C₂ to C₁₈ alkenyl group, a C₄ to C₁₈ cycloalkyl group, a C₆ to C₁₈ aryl group, or a C₇ to C₁₈ aralkyl group; alternatively, each R independently can be a C₁ to C₁₂ alkyl group, a C₂ to C₁₂ alkenyl group, a C₄ to C₁₂ cycloalkyl group, a C₆ to C₁₂ aryl group, or a C₇ to C₁₂ aralkyl group; alternatively, each R independently can be a C₁ to C₁₀ alkyl group, a C₂ to C₁₀ alkenyl group, a C₄ to C₁₀ cycloalkyl group, a C₆ to C₁₀ aryl group, or a C₇ to C₁₀ aralkyl group; or alternatively, each R independently can be a C₁ to C₅ alkyl group, a C₂ to C₅ alkenyl group, a C₅ to C₈ cycloalkyl group, a C₆ to C₈ aryl group, or a C₇ to C₈ aralkyl group.

Accordingly, in some aspects, the alkyl group which can be an R in formula (I) can be a methyl group, an ethyl group, a propyl group, a butyl group, a pentyl group, a hexyl group, a heptyl group, an octyl group, a nonyl group, a decyl group, a undecyl group, a dodecyl group, a tridecyl group, a tetradecyl group, a pentadecyl group, a hexadecyl group, a heptadecyl group, or an octadecyl group; or alternatively, a methyl group, an ethyl group, a propyl group, a butyl group, a pentyl group, a hexyl group, a heptyl group, an octyl group, a nonyl group, or a decyl group. In some aspects, the alkyl group which can be an R in formula (I) can be a methyl group, an ethyl group, a n-propyl group, an iso-propyl group, a n-butyl group, an iso-butyl group, a sec-butyl group, a tert-butyl group, a n-pentyl group, an iso-pentyl group, a sec-pentyl group, or a neopentyl group; alternatively, a methyl group, an ethyl group, an iso-propyl group, a tert-butyl group, or a neopentyl group; alternatively, a methyl group; alternatively, an ethyl group; alternatively, a n-propyl group; alternatively, an iso-propyl group; alternatively, a tert-butyl group; or alternatively, a neopentyl group.

Suitable alkenyl groups which can be an R in formula (I) can include, but are not limited to, an ethenyl group, a propenyl group, a butenyl group, a pentenyl group, a hexenyl group, a heptenyl group, an octenyl group, a nonenyl group, a decenyl group, a undecenyl group, a dodecenyl group, a tridecenyl group, a tetradecenyl group, a pentadecenyl group, a hexadecenyl group, a heptadecenyl group, or an octadecenyl group. Such alkenyl groups can be linear or branched, and the double bond can be located anywhere in the chain. In one aspect, each R in formula (I) independently can be an ethenyl group, a propenyl group, a butenyl group, a pentenyl group, a hexenyl group, a heptenyl group, an octenyl group, a nonenyl group, or a decenyl group, while in another aspect, each R in formula (I) independently can be an ethenyl group, a propenyl group, a butenyl group, a pentenyl group, or a hexenyl group. For example, an R can be an ethenyl group; alternatively, a propenyl group; alternatively, a butenyl group; alternatively, a pentenyl group; or alternatively, a hexenyl group. In yet another aspect, an R can be a terminal alkenyl group, such as a C₃ to C₁₈ terminal alkenyl group, a C₃ to C₁₂ terminal alkenyl group, or a C₃ to C₈ terminal alkenyl group. Illustrative terminal alkenyl groups can include, but are not limited to, a prop-2-en-1-yl group, a bute-3-en-1-yl group, a pent-4-en-1-yl group, a hex-5-en-1-yl group, a hept-6-en-1-yl group, an octe-7-en-1-yl group, a non-8-en-1-yl group, a dece-9-en-1-yl group, and so forth.

Each R in formula (I) independently can be a cycloalkyl group, including, but not limited to, a cyclobutyl group, a substituted cyclobutyl group, a cyclopentyl group, a substituted cyclopentyl group, a cyclohexyl group, a substituted cyclohexyl group, a cycloheptyl group, a substituted cycloheptyl group, a cyclooctyl group, or a substituted cyclooctyl group. For example, an R in formula (I) can be a cyclopentyl group, a substituted cyclopentyl group, a cyclohexyl group, or a substituted cyclohexyl group. Moreover, each R in formula (I) independently can be a cyclobutyl group or a substituted cyclobutyl group; alternatively, a cyclopentyl group or a substituted cyclopentyl group; alternatively, a cyclohexyl group or a substituted cyclohexyl group; alternatively, a cycloheptyl group or a substituted cycloheptyl group; alternatively, a cyclooctyl group or a substituted cyclooctyl group; alternatively, a cyclopentyl group; alternatively, a substituted cyclopentyl group; alternatively, a cyclohexyl group; or alternatively, a substituted cyclohexyl group. Substituents which can be utilized for the substituted cycloalkyl group are independently disclosed herein and can be utilized without limitation to further describe the substituted cycloalkyl group which can be an R in formula (I).

In some aspects, the aryl group which can be an R in formula (I) can be a phenyl group, a substituted phenyl group, a naphthyl group, or a substituted naphthyl group. In an aspect, the aryl group can be a phenyl group or a substituted phenyl group; alternatively, a naphthyl group or a substituted naphthyl group; alternatively, a phenyl group or a naphthyl group; alternatively, a substituted phenyl group or a substituted naphthyl group; alternatively, a phenyl group; or alternatively, a naphthyl group. Substituents which can be utilized for the substituted phenyl groups or substituted naphthyl groups are independently disclosed herein and can be utilized without limitation to further describe the substituted phenyl groups or substituted naphthyl groups which can be an Rin formula (I).

In an aspect, the substituted phenyl group which can be an R in formula (I) can be a 2-substituted phenyl group, a 3-substituted phenyl group, a 4-substituted phenyl group, a 2,4-disubstituted phenyl group, a 2,6-disubstituted phenyl group, a 3,5-disubstituted phenyl group, or a 2,4,6-trisubstituted phenyl group. In other aspects, the substituted phenyl group can be a 2-substituted phenyl group, a 4-substituted phenyl group, a 2,4-disubstituted phenyl group, or a 2,6-disubstituted phenyl group; alternatively, a 3-substituted phenyl group or a 3,5-disubstituted phenyl group; alternatively, a 2-substituted phenyl group or a 4-substituted phenyl group; alternatively, a 2,4-disubstituted phenyl group or a 2,6-disubstituted phenyl group; alternatively, a 2-substituted phenyl group; alternatively, a 3-substituted phenyl group; alternatively, a 4-substituted phenyl group; alternatively, a 2,4-disubstituted phenyl group; alternatively, a 2,6-disubstituted phenyl group; alternatively, a 3,5-disubstituted phenyl group; or alternatively, a 2,4,6-trisubstituted phenyl group. Substituents which can be utilized for these specific substituted phenyl groups are independently disclosed herein and can be utilized without limitation to further describe these substituted phenyl groups which can be an R group(s) in formula (I).

In some aspects, the aralkyl group which can be an R group in formula (I) can be a benzyl group or a substituted benzyl group. In an aspect, the aralkyl group can be a benzyl group or, alternatively, a substituted benzyl group. Substituents which can be utilized for the substituted aralkyl group are independently disclosed herein and can be utilized without limitation to further describe the substituted aralkyl group which can be an R group(s) in formula (I).

In an aspect, each non-hydrogen substituent(s) for the substituted cycloalkyl group, substituted aryl group, or substituted aralkyl group which can be an R in formula (I) independently can be a C₁ to C₁₈ hydrocarbyl group; alternatively, a C₁ to C₈ hydrocarbyl group; or alternatively, a C₁ to C₅ hydrocarbyl group. Specific hydrocarbyl groups are independently disclosed herein and can be utilized without limitation to further describe the substituents of the substituted cycloalkyl groups, substituted aryl groups, or substituted aralkyl groups which can be an R in formula (I). For instance, the hydrocarbyl substituent can be an alkyl group, such as a methyl group, an ethyl group, a n-propyl group, an isopropyl group, a n-butyl group, a sec-butyl group, an isobutyl group, a tert-butyl group, a n-pentyl group, a 2-pentyl group, a 3-pentyl group, a 2-methyl-1-butyl group, a tert-pentyl group, a 3-methyl-1-butyl group, a 3-methyl-2-butyl group, or a neo-pentyl group, and the like. Furthermore, the hydrocarbyl substituent can be a benzyl group, a phenyl group, a tolyl group, or a xylyl group, and the like.

A hydrocarboxy group is used generically herein to include, for instance, alkoxy, aryloxy, aralkoxy, -(alkyl, aryl, or aralkyl)-O-(alkyl, aryl, or aralkyl) groups, and —O(CO)-(hydrogen or hydrocarbyl) groups, and these groups can comprise up to 36 carbon atoms (e.g., C₁ to C₃₆, C₁ to C₁₈, C₁ to C₁₀, or C₁ to C₈ hydrocarboxy groups). Illustrative and non-limiting examples of hydrocarboxy groups which can be an R in formula (I) can include, but are not limited to, a methoxy group, an ethoxy group, an n-propoxy group, an isopropoxy group, an n-butoxy group, a sec-butoxy group, an isobutoxy group, a tert-butoxy group, an n-pentoxy group, a 2-pentoxy group, a 3-pentoxy group, a 2-methyl-1-butoxy group, a tert-pentoxy group, a 3-methyl-1-butoxy group, a 3-methyl-2-butoxy group, a neo-pentoxy group, a phenoxy group, a toloxy group, a xyloxy group, a 2,4,6-trimethylphenoxy group, a benzoxy group, an acetylacetonate group (acac), a formate group, an acetate group, a stearate group, an oleate group, a benzoate group, and the like. In an aspect, the hydrocarboxy group which can be an R in formula (I) can be a methoxy group; alternatively, an ethoxy group; alternatively, an n-propoxy group; alternatively, an isopropoxy group; alternatively, an n-butoxy group; alternatively, a sec-butoxy group; alternatively, an isobutoxy group; alternatively, a tert-butoxy group; alternatively, an n-pentoxy group; alternatively, a 2-pentoxy group; alternatively, a 3-pentoxy group; alternatively, a 2-methyl-1-butoxy group; alternatively, a tert-pentoxy group; alternatively, a 3-methyl-1-butoxy group, alternatively, a 3-methyl-2-butoxy group; alternatively, a neo-pentoxy group; alternatively, a phenoxy group; alternatively, a toloxy group; alternatively, a xyloxy group; alternatively, a 2,4,6-trimethylphenoxy group; alternatively, a benzoxy group; alternatively, an acetylacetonate group; alternatively, a formate group; alternatively, an acetate group; alternatively, a stearate group; alternatively, an oleate group; or alternatively, a benzoate group.

In accordance with some aspects disclosed herein, each R independently can be a C₁ to C₃₆ hydrocarbylsilyl group; alternatively, a C₁ to C₂₄ hydrocarbylsilyl group; alternatively, a C₁ to C₁₈ hydrocarbylsilyl group; or alternatively, a C₁ to C₈ hydrocarbylsilyl group. In an aspect, each hydrocarbyl (one or more) of the hydrocarbylsilyl group can be any hydrocarbyl group disclosed herein (e.g., a C₁ to C₅ alkyl group, a C₂ to C₅ alkenyl group, a C₅ to C₈ cycloalkyl group, a C₆ to C₈ aryl group, a C₇ to C₈ aralkyl group, etc.). As used herein, hydrocarbylsilyl is intended to cover (mono)hydrocarbylsilyl (—SiH₂R), dihydrocarbylsilyl (—SiHR₂), and trihydrocarbylsilyl (—SiR₃) groups, with R being a hydrocarbyl group. In one aspect, the hydrocarbylsilyl group can be a C₃ to C₃₆ or a C₃ to C₁₈ trihydrocarbylsilyl group, such as, for example, a trialkylsilyl group or a triphenylsilyl group. Illustrative and non-limiting examples of hydrocarbylsilyl groups which can be an R group(s) in formula (I) can include, but are not limited to, trimethylsilyl, triethylsilyl, tripropylsilyl (e.g., triisopropylsilyl), tributylsilyl, tripentylsilyl, triphenylsilyl, allyldimethylsilyl, and the like.

In formula (I), each R independently can be a C₁ to C₃₆ halogenated hydrocarbyl group, a C₁ to C₁₈ halogenated hydrocarbyl group, a C₁ to C₁₂ halogenated hydrocarbyl group, or a C₁ to C₈ halogenated hydrocarbyl group, where the halogenated hydrocarbyl group indicates the presence of one or more halogen atoms replacing an equivalent number of hydrogen atoms in the hydrocarbyl group. The halogenated hydrocarbyl group often can be a halogenated alkyl group, a halogenated alkenyl group, a halogenated cycloalkyl group, a halogenated aryl group, or a halogenated aralkyl group. Representative and non-limiting halogenated hydrocarbyl groups include pentafluorophenyl, trifluoromethyl (CF₃), and the like.

In one aspect, each R independently can be H, a halide, or a C₁ to C₃₆ hydrocarbyl group, hydrocarboxy group, or hydrocarbylsilyl group, while in another aspect, each R independently can be H, or a C₁ to C₁₈ hydrocarbyl group or halogenated hydrocarbyl group. In yet another aspect, each R independently can be H, Cl, Br, a C₁ to C₁₈ hydrocarbyl group, a C₁ to C₁₈ hydrocarboxy group, a C₁ to C₁₈ hydrocarbylsilyl group, or a C₁ to C₁₈ halogenated hydrocarbyl group. In still another aspect, each R independently can be H, a halide, a hydrocarbyl group, an alkoxy, an aryloxy, acetylacetonate, formate, acetate, stearate, oleate, benzoate, or a trihydrocarbylsilyl group. In these and other aspects, the alkoxy, aryloxy, hydrocarbyl, and trihydrocarbylsilyl can be a C₁ to C₃₆, a C₁ to C₁₈, a C₁ to C₁₂, or a C₁ to C₈ alkoxy, aryloxy, hydrocarbyl, and trihydrocarbylsilyl.

Moreover, each R independently can be, in certain aspects, H, methyl, ethyl, propyl, butyl, pentyl, hexyl, heptyl, octyl, nonyl, decyl, ethenyl, propenyl, butenyl, pentenyl, hexenyl, heptenyl, octenyl, nonenyl, decenyl, phenyl, tolyl, benzyl, naphthyl, trimethylsilyl, triisopropylsilyl, triphenylsilyl, allyldimethylsilyl, or a halide.

Any suitable number of non-hydrogen R groups can be present in formula (I). For instance, in one aspect, each R is a H. However, in other aspects, one R group can be a non-hydrogen substituent, two R groups independently can be non-hydrogen substituents, three R groups independently can be non-hydrogen substituents, four R groups independently can be non-hydrogen substituents, five R groups independently can be non-hydrogen substituents, six R groups independently can be non-hydrogen substituents, and so forth. As disclosed, each R in formula (I) can be either the same or a different substituent group. Moreover, each non-hydrogen substituent can be at any position in formula (I) that conforms with the rules of chemical valence.

In formula (I), each X independently can be any suitable halide. Thus, each X independently can be F, Cl, Br, or I. In one aspect, each X independently can be Cl or Br, while in another aspect, each X can be Cl, and in yet another aspect, each X can be Br.

R¹ and R² in formula (I), independently, can be any suitable substituent, or R¹ and R² can be linked to form any suitable ring or ring system. For instance, the substituent selections for R¹ and R² in formula (I) can encompass the same as those described herein for each R in formula (I). Therefore, R¹ and R² independently can be H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group; alternatively, R¹ and R² independently can be H, a halide, a C₁ to C₁₈ hydrocarbyl group, a C₁ to C₁₈ halogenated hydrocarbyl group, a C₁ to C₁₈ hydrocarboxy group, or a C₁ to C₁₈ hydrocarbylsilyl group; alternatively, R¹ and R² independently can be H, a halide, a C₁ to C₈ hydrocarbyl group, a C₁ to C₈ halogenated hydrocarbyl group, a C₁ to C₈ hydrocarboxy group, or a C₁ to C₈ hydrocarbylsilyl group; alternatively, R¹ and R² independently can be H, a halide, or a C₁ to C₁₈ hydrocarbyl group; alternatively, R¹ and R² independently can be H or a C₁ to C₁₈ hydrocarbyl group; alternatively, R¹ and R² independently can be H, a halide, or a C₁ to C₈ alkyl group; or alternatively, R¹ and R² independently can be H or a C₁ to C₈ alkyl group.

Alternatively, R¹ and R² can be linked to form a ring or ring system. In one aspect, R¹ and R² can be linked to form a cycloalkyl ring or ring system, while in another aspect, R¹ and R² can be linked to form an aromatic ring or ring system. Such rings and ring systems are well known to those of skill in art, and are disclosed, for example, in U.S. Pat. No. 6,291,608 and U.S. Patent Publication No. 2014/0088319, which are incorporated herein by reference in their entirety.

Illustrative and non-limiting examples of alpha-diimine nickel halide complexes having formula (I) and/or suitable for use as in catalyst compositions of this invention can include the following three representative alpha-diimine nickel bromide formulas:

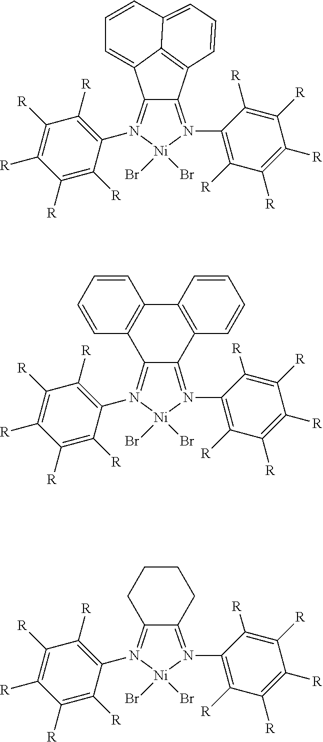

In these formulas, each R independently can be any substituent disclosed herein, such as H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group; alternatively, each R independently can be H, a halide, a C₁ to C₈ hydrocarbyl group, a C₁ to C₈ halogenated hydrocarbyl group, a C₁ to C₈ hydrocarboxy group, or a C₁ to C₈ hydrocarbylsilyl group; alternatively, each R independently can be H or a C₁ to C₁₈ hydrocarbyl group; or alternatively, each R independently can be H or a C₁ to C₈ alkyl group.

The alpha-diimine nickel halide complex is not limited solely to alpha-diimine nickel halide complexes such as described above. Other suitable alpha-diimine nickel halide complexes are disclosed in U.S. Pat. No. 6,291,608 and U.S. Patent Publication No. 2014/0088319, which are incorporated herein by reference in their entirety.

### Chemically-Treated Solid Oxides

In the catalyst compositions and oligomerization processes disclosed herein, any suitable chemically-treated solid oxide can be employed, whether one chemically-treated solid oxide or a mixture or combination of two or more different chemically-treated solid oxides. In one aspect, the chemically-treated solid oxide can comprise a solid oxide treated with an electron-withdrawing anion. Alternatively, in another aspect, the chemically-treated solid oxide can comprise a solid oxide treated with an electron-withdrawing anion, the solid oxide containing a Lewis-acidic metal ion. Non-limiting examples of suitable chemically-treated solid oxides are disclosed in, for instance, U.S. Pat. Nos. 7,294,599, 7,601,665, 7,884,163, 8,309,485, 8,623,973, 8,703,886, and 9,023,959, which are incorporated herein by reference in their entirety.

The solid oxide can encompass oxide materials such as alumina, “mixed oxides” thereof such as silica-alumina, coatings of one oxide on another, and combinations and mixtures thereof. The mixed oxides such as silica-alumina can be single or multiple chemical phases with more than one metal combined with oxygen to form the solid oxide. Examples of mixed oxides that can be used to form a chemically-treated solid oxide, either singly or in combination, can include, but are not limited to, silica-alumina, silica-titania, silica-zirconia, alumina-titania, alumina-zirconia, zinc-aluminate, alumina-boria, silica-bona, aluminophosphate-silica, and titania-zirconia. The solid oxide used herein also can encompass oxide materials such as silica-coated alumina, as described in U.S. Pat. No. 7,884,163 (e.g., Sasol Siral® 28, Sasol Siral® 40, etc.)

Accordingly, in one aspect, the solid oxide can comprise silica, alumina, silica-alumina, silica-coated alumina, aluminum phosphate, aluminophosphate, heteropolytungstate, titania, silica-titania, zirconia, silica-zirconia, magnesia, boria, zinc oxide, any mixed oxide thereof, or any combination thereof. In another aspect, the solid oxide can comprise alumina, silica-alumina, silica-coated alumina, aluminum phosphate, aluminophosphate, heteropolytungstate, titania, silica-titania, zirconia, silica-zirconia, magnesia, boria, or zinc oxide, as well as any mixed oxide thereof, or any mixture thereof. The solid oxides contemplated herein can have any suitable surface area, pore volume, and particle size, as would be recognized by those of skill in the art. In another aspect, the solid oxide can comprise silica, alumina, titania, zirconia, magnesia, boria, zinc oxide, any mixed oxide thereof, or any combination thereof. In yet another aspect, the solid oxide can comprise silica-alumina, silica-coated alumina, silica-titania, silica-zirconia, alumina-boria, or any combination thereof. In still another aspect, the solid oxide can comprise silica, alumina, silica-alumina, silica-coated alumina, or any mixture thereof; alternatively, silica; alternatively, alumina; alternatively, silica-alumina; or alternatively, silica-coated alumina.

The silica-coated alumina solid oxide materials which can be used in the catalyst compositions and oligomerization processes often are alumina-rich, for instance, the weight ratio of alumina to silica (alumina: silica) in the silica-coated alumina can be in a range from about 1.05:1 to about 50:1, from about 1.1:1 to about 50:1, or from about 1.2:1 to about 50:1. In one aspect, the weight ratio of alumina:silica in the silica-coated alumina can be in a range from about 1.05:1 to about 25:1; alternatively, from about 1.05:1 to about 12:1; alternatively, from about 1.05:1 to about 6:1; or alternatively, from about 1.05:1 to about 4:1. In another aspect, the weight ratio of alumina:silica in the silica-coated alumina can be in a range from about 1.1:1 to about 25:1; alternatively, from about 1.1:1 to about 12:1; alternatively, from about 1.1:1 to about 7:1; or alternatively, from about 1.1:1 to about 3:1. In yet another aspect, the weight ratio of alumina:silica in the silica-coated alumina can be in a range from about 1.2:1 to about 25:1; alternatively, from about 1.2:1 to about 12:1; alternatively, from about 1.2:1 to about 6:1; alternatively, from about 1.2:1 to about 4:1; or alternatively, from about 1.2:1 to about 3:1. In still another aspect, the weight ratio of alumina:silica in the silica-coated alumina can be in a range from about 1.3:1 to about 25:1; alternatively, from about 1.3:1 to about 12:1; alternatively, from about 1.3:1 to about 6:1; alternatively, from about 1.3:1 to about 4:1; or alternatively, from about 1.3:1 to about 3:1.

The electron-withdrawing component used to treat the solid oxide can be any component that can increase the Lewis or Bronsted acidity of the solid oxide upon treatment (as compared to the solid oxide that is not treated with at least one electron-withdrawing anion). According to one aspect, the electron-withdrawing component can be an electron-withdrawing anion derived from a salt, an acid, or other compound, such as a volatile organic compound, that serves as a source or precursor for that anion. Examples of electron-withdrawing anions can include, but are not limited to, sulfate, bisulfate, fluoride, chloride, bromide, iodide, fluorosulfate, fluoroborate, phosphate, acetate, fluorophosphate, trifluoroacetate, triflate, fluorozirconate, fluorotitanate, phospho-tungstate, tungstate, and molybdate, including mixtures and combinations thereof. In addition, other ionic or non-ionic compounds that serve as sources for these electron-withdrawing anions also can be employed. It is contemplated that the electron-withdrawing anion can be, or can comprise, fluoride, chloride, bromide, phosphate, triflate, bisulfate, or sulfate, or any combination thereof, in some aspects provided herein. In other aspects, the electron-withdrawing anion can comprise sulfate, bisulfate, fluoride, chloride, bromide, iodide, fluorosulfate, fluoroborate, phosphate, acetate, fluorophosphate, trifluoroacetate, triflate, fluorozirconate, fluorotitanate, or combinations thereof. Yet, in other aspects, the electron-withdrawing anion can comprise sulfate, fluoride, chloride, or combinations thereof; alternatively, sulfate; alternatively, fluoride and chloride; or alternatively, fluoride.

The chemically-treated solid oxide generally can contain from about 1 to about 30 wt. % of the electron-withdrawing anion, based on the weight of the chemically-treated solid oxide. In particular aspects provided herein, the chemically-treated solid oxide can contain from about 1 to about 20 wt. %, from about 2 to about 20 wt. %, from about 3 to about 20 wt. %, from about 2 to about 15 wt. %, from about 3 to about 15 wt. %, from about 3 to about 12 wt. %, from about 4 to about 10 wt. %, or from about 5 to about 9 wt. %, of the electron-withdrawing anion, based on the total weight of the chemically-treated solid oxide.

In an aspect, the chemically-treated solid oxide can comprise fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, or phosphated silica-coated alumina, as well as any mixture or combination thereof. In another aspect, the chemically-treated solid oxide employed in the catalyst compositions and oligomerization processes described herein can be, or can comprise, a fluorided solid oxide and/or a sulfated solid oxide, non-limiting examples of which can include fluorided alumina, sulfated alumina, fluorided silica-alumina, sulfated silica-alumina, fluorided silica-zirconia, fluorided silica-coated alumina, fluorided-chlorided silica-coated alumina, or sulfated silica-coated alumina, as well as combinations thereof. In yet another aspect, the chemically-treated solid oxide can comprise fluorided alumina; alternatively, chlorided alumina; alternatively, sulfated alumina; alternatively, fluorided silica-alumina;

alternatively, sulfated silica-alumina; alternatively, fluorided silica-zirconia; alternatively, chlorided silica-zirconia; alternatively, sulfated silica-coated alumina; alternatively, fluorided-chlorided silica-coated alumina; or alternatively, fluorided silica-coated alumina. In some aspects, the chemically-treated solid oxide can comprise a fluorided solid oxide, while in other aspects, the chemically-treated solid oxide can comprise a sulfated solid oxide.

Various processes can be used to form chemically-treated solid oxides useful in the present invention. Methods of contacting the solid oxide with the electron-withdrawing component, suitable electron withdrawing components and addition amounts, impregnation with metals or metal ions (e.g., zinc, nickel, vanadium, titanium, silver, copper, gallium, tin, tungsten, molybdenum, zirconium, or combinations thereof), various calcining procedures and conditions (e.g., calcining temperatures in a range from about 300° C. to about 900° C., from about 400° C. to about 800° C., or from about 500° C. to about 700° C.), calcination times (e.g., calcination times in a range from about 1 minute to about 24 hours, from about 5 minutes to about 10 hours, or from about 20 minutes to about 6 hours), calcination equipment (e.g., calcination equipment such as a rotary kiln, muffle furnace, or fluidized bed, among other methods of conveying heat), and calcination atmospheres (e.g., dry or humid calcination atmospheres, oxidizing calcination atmospheres such as air or oxygen, reducing calcination atmospheres such as carbon monoxide or hydrogen, or non-reactive calcination atmospheres like nitrogen or argon) are disclosed in, for example, U.S. Pat. Nos. 6,107,230, 6,165,929, 6,294,494, 6,300,271, 6,316,553, 6,355,594, 6,376,415, 6,388,017, 6,391,816, 6,395,666, 6,524,987, 6,548,441, 6,548,442, 6,576,583, 6,613,712, 6,632,894, 6,667,274, 6,750,302, 7,294,599, 7,601,665, 7,884,163, and 8,309,485, which are incorporated herein by reference in their entirety. Other suitable processes and procedures for preparing chemically-treated solid oxides (e.g., chemically-treated silica-coated aluminas, such as fluorided silica-coated alumina) are well known to those of skill in the art.

### CO-CATALYSTS

In certain aspects directed to catalyst compositions containing a co-catalyst, the co-catalyst can comprise a metal hydrocarbyl compound, examples of which can include non-halide metal hydrocarbyl compounds, metal hydrocarbyl halide compounds, non-halide metal alkyl compounds, metal alkyl halide compounds, and so forth. The hydrocarbyl group (or alkyl group) can be any hydrocarbyl (or alkyl) group disclosed herein. Moreover, in some aspects, the metal of the metal hydrocarbyl can be a group 1, 2, 11, 12, 13, or 14 metal; alternatively, a group 13 or 14 metal; or alternatively, a group 13 metal. Hence, in some aspects, the metal of the metal hydrocarbyl (non-halide metal hydrocarbyl or metal hydrocarbyl halide) can be lithium, sodium, potassium, rubidium, cesium, beryllium, magnesium, calcium, strontium, barium, zinc, cadmium, boron, aluminum, or tin; alternatively, lithium, sodium, potassium, magnesium, calcium, zinc, boron, aluminum, or tin; alternatively, lithium, sodium, or potassium; alternatively, magnesium or calcium; alternatively, lithium; alternatively, sodium; alternatively, potassium; alternatively, magnesium; alternatively, calcium; alternatively, zinc; alternatively, boron; alternatively, aluminum; or alternatively, tin. In some aspects, the metal hydrocarbyl or metal alkyl, with or without a halide, can comprise a lithium hydrocarbyl or alkyl, a magnesium hydrocarbyl or alkyl, a boron hydrocarbyl or alkyl, a zinc hydrocarbyl or alkyl, or an aluminum hydrocarbyl or alkyl.

In particular aspects directed to catalyst compositions containing a co-catalyst (i.e., the activator can comprise a chemically-treated solid oxide), the co-catalyst can comprise an aluminoxane compound, an organoboron or organoborate compound, an ionizing ionic compound, an organoaluminum compound, an organozinc compound, an organomagnesium compound, or an organolithium compound, and this can include any combinations of these materials. In one aspect, the co-catalyst can comprise an organoaluminum compound. In another aspect, the co-catalyst can comprise an aluminoxane compound, an organoboron or organoborate compound, an ionizing ionic compound, an organozinc compound, an organomagnesium compound, an organolithium compound, or any combination thereof. In yet another aspect, the co-catalyst can comprise an aluminoxane compound; alternatively, an organoboron or organoborate compound; alternatively, an ionizing ionic compound; alternatively, an organozinc compound; alternatively, an organomagnesium compound; or alternatively, an organolithium compound.

Specific non-limiting examples of suitable organoaluminum compounds can include trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum, diisobutylaluminum hydride, diethylaluminum ethoxide, diethylaluminum chloride, and the like, or combinations thereof. Representative and non-limiting examples of aluminoxanes can include methylaluminoxane, modified methylaluminoxane, ethylaluminoxane, n-propylaluminoxane, iso-propylaluminoxane, n-butylaluminoxane, t-butylaluminoxane, sec-butylaluminoxane, iso-butylaluminoxane, 1-pentylaluminoxane, 2-pentylaluminoxane, 3-pentylaluminoxane, isopentyl-aluminoxane, neopentylaluminoxane, and the like, or any combination thereof. Representative and non-limiting examples of organoboron/organoborate compounds can include N,N-dimethylanilinium tetrakis(pentafluorophenyl)borate, triphenylcarbenium tetrakis(pentafluorophenyl)borate, lithium tetrakis(pentafluorophenyl)borate, N,N-dimethylanilinium tetrakis [3,5-bis(trifluoro-methyl)phenyl]borate, triphenylcarbenium tetrakis [3,5-bis(trifluoromethyl)phenyl]borate, tris(pentafluorophenyl)boron, tris[3,5-bis(trifluoromethyl)phenyl]boron, and the like, or mixtures thereof.

Examples of ionizing ionic compounds can include, but are not limited to, the following compounds: tri(n-butyl)ammonium tetrakis(p-tolyl)borate, tri(n-butyl) ammonium tetrakis(m-tolyl)borate, tri(n-butyl)ammonium tetrakis(2,4-dimethylphenyl)borate, tri(n-butyl)ammonium tetrakis(3,5-dimethylphenyl)borate, tri(n-butyl)ammonium tetrakis [3,5-bis(trifluoro-methyl)phenyl]borate, tri(n-butyl)ammonium tetrakis(pentafluorophenyl)borate, N,N-dimethylanilinium tetrakis(p-tolyl)borate, N,N-dimethylanilinium tetrakis(m-tolyl)borate, N,N-dimethylanilinium tetrakis(2,4-dimethylphenyl)borate, N,N-dimethylanilinium tetrakis(3,5-dimethyl-phenyl)borate, N,N-dimethylanilinium tetrakis [3,5-bis(trifluoromethyl)phenyl]borate, N,N-dimethylanilinium tetrakis(pentafluorophenyl)borate, triphenylcarbenium tetrakis(p-tolyl)borate, triphenylcarbenium tetrakis(m-tolyl)borate, triphenylcarbenium tetrakis(2,4-dimethylphenyl)borate, triphenylcarbenium tetrakis(3,5-dimethylphenyl)borate, triphenylcarbenium tetrakis [3,5-bis(trifluoro-methyl)phenyl]borate, triphenylcarbenium tetrakis(pentafluorophenyl)borate, tropylium tetrakis(p-tolyl)borate, tropylium tetrakis(m-tolyl)borate, tropylium tetrakis(2,4-dimethylphenyl)borate, tropylium tetrakis(3,5-dimethylphenyl)borate, tropylium tetrakis [3,5-bis(trifluoro-methyl)phenyl]borate, tropylium tetrakis(pentafluorophenyl) borate, lithium tetrakis(pentafluorophenyl)borate, lithium tetraphenylborate, lithium tetrakis(p-tolyl)borate, lithium tetrakis(m-tolyl)borate, lithium tetrakis(2,4-dimethylphenyl)borate, lithium tetrakis(3,5-dimethylphenyl)borate, lithium tetrafluoroborate, sodium tetrakis(pentafluorophenyl)borate, sodium tetraphenylborate, sodium tetrakis(p-tolyl)borate, sodium tetrakis(m-tolyl)borate, sodium tetrakis(2,4-dimethylphenyl)borate, sodium tetrakis(3,5-dimethylphenyl)borate, sodium tetrafluoroborate, potassium tetrakis(pentafluorophenyl)borate, potassium tetraphenylborate, potassium tetrakis(p-tolyl)borate, potassium tetrakis(m-tolyl)borate, potassium tetrakis(2,4-dimethylphenyl)borate, potassium tetrakis(3,5-dimethylphenyl)borate, potassium tetrafluoroborate, lithium tetrakis(pentafluorophenyl)aluminate, lithium tetraphenylaluminate, lithium tetrakis(p-tolyl)aluminate, lithium tetrakis(m-tolyl)aluminate, lithium tetrakis(2,4-dimethylphenyl)aluminate, lithium tetrakis(3,5-dimethylphenyl)aluminate, lithium tetrafluoroaluminate, sodium tetrakis(pentafluorophenyl)aluminate, sodium tetraphenylaluminate, sodium tetrakis(p-tolyl)-aluminate, sodium tetrakis(m-tolyl)aluminate, sodium tetrakis(2,4-dimethylphenyl)aluminate, sodium tetrakis(3,5-dimethylphenyl)aluminate, sodium tetrafluoroaluminate, potassium tetrakis(pentafluorophenyl)aluminate, potassium tetraphenylaluminate, potassium tetrakis(p-tolyl)aluminate, potassium tetrakis(m-tolyl)aluminate, potassium tetrakis(2,4-dimethylphenyl)aluminate, potassium tetrakis (3,5-dimethylphenyl)aluminate, potassium tetrafluoroaluminate, and the like, or combinations thereof.

Exemplary organozinc compounds which can be used as co-catalysts can include, but are not limited to, dimethylzinc, diethylzinc, dipropylzinc, dibutylzinc, dineopentylzinc, di(trimethylsily)zinc, di(triethylsily)zinc, di(triisoproplysily)zinc, di(triphenylsily)zinc, di(allyldimethylsilyl)zinc, di(trimethylsilylmethyl)zinc, and the like, or combinations thereof.

Similarly, exemplary organomagnesium compounds can include, but are not limited to, dimethylmagnesium, diethylmagnesium, dipropylmagnesium, dibutylmagnesium, dineopentylmagnesium, di(trimethylsilylmethyl)magnesium, methylmagnesium chloride, ethylmagnesium chloride, propylmagnesium chloride, butylmagnesium chloride, neopentylmagnesium chloride, trimethylsilylmethylmagnesium chloride, methylmagnesium bromide, ethylmagnesium bromide, propylmagnesium bromide, butylmagnesium bromide, neopentylmagnesium bromide, trimethylsilylmethylmagnesium bromide, methylmagnesium iodide, ethylmagnesium iodide, propylmagnesium iodide, butylmagnesium iodide, neopentylmagnesium iodide, trimethylsilylmethylmagnesium iodide, methylmagnesium ethoxide, ethylmagnesium ethoxide, propylmagnesium ethoxide, butylmagnesium ethoxide, neopentylmagnesium ethoxide, trimethylsilylmethylmagnesium ethoxide, methylmagnesium propoxide, ethylmagnesium propoxide, propylmagnesium propoxide, butylmagnesium propoxide, neopentylmagnesium propoxide, trimethylsilylmethylmagnesium propoxide, methylmagnesium phenoxide, ethylmagnesium phenoxide, propylmagnesium phenoxide, butylmagnesium phenoxide, neopentylmagnesium phenoxide, trimethylsilylmethylmagnesium phenoxide, and the like, or any combinations thereof.

Likewise, exemplary organolithium compounds can include, but are not limited to, methyllithium, ethyllithium, propyllithium, butyllithium (e.g., t-butyllithium), neopentyllithium, trimethylsilylmethyllithium, phenyllithium, tolyllithium, xylyllithium, benzyllithium, (dimethylphenyl)methyllithium, allyllithium, and the like, or combinations thereof.

Co-catalysts that can be used in the catalyst compositions of this invention are not limited to the co-catalysts described above. Other suitable co-catalysts are well known to those of skill in the art including, for example, those disclosed in U.S. Pat. Nos. 3,242,099, 4,794,096, 4,808,561, 5,576,259, 5,807,938, 5,919,983, 7,294,599 7,601,665, 7,884,163, 8,114,946, and 8,309,485, which are incorporated herein by reference in their entirety.

### Catalyst Compositions

In some aspects, the present invention can employ catalyst compositions containing an alpha-diimine nickel halide complex, a chemically-treated solid oxide, and a co-catalyst. These catalyst compositions can be utilized to reduce the light fraction of a propylene oligomer feedstock to produce higher propylene oligomer products, for a variety of end-use applications. The alpha-diimine nickel halide complex is discussed hereinabove. In aspects of the present invention, it is contemplated that the catalyst composition can contain more than one alpha-diimine nickel halide complex. Further, additional catalytic compounds—other than those specified as an alpha-diimine nickel halide complex—can be employed in the catalyst compositions and/or the oligomerization processes, provided that the additional catalytic compound(s) does not detract from the advantages disclosed herein. Additionally, more than one chemically-treated solid oxide and/or more than one co-catalyst also can be utilized. Chemically-treated solid oxides and co-catalysts (e.g., organoaluminum compounds) useful in the present invention are disclosed hereinabove.

Accordingly, a catalyst composition of this invention can comprise an alpha-diimine nickel halide complex, a chemically-treated solid oxide, and an organoaluminum compound. For instance, the chemically-treated solid oxide can comprise (or consist essentially of, or consist of) fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, and the like, or combinations thereof; alternatively, the chemically-treated solid oxide can comprise (or consist essentially of, or consist of) a fluorided solid oxide and/or a sulfated solid oxide. Additionally, the organoaluminum compound can comprise (or consist essentially of, or consist of) trimethylaluminum, triethylaluminum, tri-n-propylaluminum, tri-n-butylaluminum, triisobutylaluminum, tri-n-hexylaluminum, tri-n-octylaluminum, diisobutylaluminum hydride, diethylaluminum ethoxide, diethylaluminum chloride, and the like, or combinations thereof. Therefore, a catalyst composition consistent with aspects of the invention can comprise (or consist essentially of, or consist of) an alpha-diimine nickel halide complex, sulfated alumina (or fluorided silica-alumina, or fluorided silica-coated alumina, or fluorided-chlorided silica-coated alumina), and triethylaluminum (or triisobutylaluminum).

In another aspect of the present invention, a catalyst composition is provided which comprises an alpha-diimine nickel halide complex, a chemically-treated solid oxide, and an organoaluminum compound, wherein this catalyst composition is substantially free of aluminoxanes, organoboron or organoborate compounds, ionizing ionic compounds, and/or other similar materials; alternatively, substantially free of aluminoxanes; alternatively, substantially free of organoboron or organoborate compounds; or alternatively, substantially free of ionizing ionic compounds. In these aspects, the catalyst composition has catalyst activity in the absence of these additional materials. For example, a catalyst composition of the present invention can consist essentially of an alpha-diimine nickel halide complex, a chemically-treated solid oxide, and an organoaluminum compound, wherein no other materials are present in the catalyst composition which would increase/decrease the activity of the catalyst composition by more than 10% or 15% from the catalyst activity of the catalyst composition in the absence of said materials.

However, in other aspects of this invention, these co-catalysts can be employed. For example, a catalyst composition comprising an alpha-diimine nickel halide complex and a chemically-treated solid oxide can further comprise an optional co-catalyst. Suitable co-catalysts in this aspect can include, but are not limited to, aluminoxane compounds, organoboron or organoborate compounds, ionizing ionic compounds, and the like, or any combination thereof; or alternatively, organoaluminum compounds, organozinc compounds, organomagnesium compounds, organolithium compounds, or any combination thereof. More than one co-catalyst can be present in the catalyst composition.

This invention further encompasses methods of making these catalyst compositions, such as, for example, by contacting the respective catalyst components—e.g., the alpha-diimine nickel halide complex, the chemically-treated solid oxide, and the co-catalyst—in any order or sequence.

In some aspects of this invention, the weight ratio of the co-catalyst (e.g., an organoaluminum compound) to the alpha-diimine nickel halide complex can be in a range from about 1:10 to about 500:1; alternatively, from about 1:10 to about 100:1; alternatively, from about 1:1 to about 100:1; alternatively, from about 5:1 to about 75:1; or alternatively, from about 10:1 to about 50:1.

In some aspects of this invention, the weight ratio of chemically-treated solid oxide to the alpha-diimine nickel halide complex can be in a range from about 2:1 to about 5000:1. In another aspect, this weight ratio can be in a range from about 5:1 to about 1000:1, from about 5:1 to about 500:1, from about 10:1 to about 800:1, or from about 10:1 to about 300:1. Yet, in another aspect, the weight ratio of the chemically-treated solid oxide to the alpha-diimine nickel halide complex can be in a range from about 10:1 to about 200:1, from about 10:1 to about 150:1, or from about 20:1 to about 100:1.

In accordance with the present invention, a process is provided that comprises contacting an olefin feedstock comprising (or consisting essentially of, or consisting of) C₆ to C₂₇ propylene oligomers with a catalyst composition comprising (or consisting essentially of, or consisting of) an alpha-diimine nickel halide complex, a chemically-treated solid oxide, and an optional co-catalyst, thereby forming an oligomer composition. While not being limited thereto, the weight ratio of the olefin feedstock to the alpha-diimine nickel halide complex often ranges from about 10:1 to about 10,000:1. For instance, the weight ratio can be in a range from about 50:1 to about 8000:1, from about 50:1 to about 5000:1, or from about 100:1 to about 5000:1, in some aspects of this invention, and the weight ratio can be in a range from about 100:1 to about 2500:1, from about 200:1 to about 2000:1, or from about 250:1 to about 1500:1, in other aspects of this invention.

Additionally or alternatively, the weight ratio of the C₆ to C₂₇ propylene oligomers to the alpha-diimine nickel halide complex often can range from about 10:1 to about 10,000:1, but is not limited thereto. In particular aspects of this invention, the weight ratio of the C₆ to C₂₇ propylene oligomers to the alpha-diimine nickel halide complex can fall within a range from about 50:1 to about 8000:1, from about 50:1 to about 5000:1, from about 100:1 to about 5000:1, from about 100:1 to about 2500:1, from about 200:1 to about 2000:1, or from about 250:1 to about 1500:1.

### Oligomerization Processes

Aspects of this invention are directed to processes for reducing the light oligomer content of propylene oligomer oils, the production of an oligomer composition, and the formation of an oligomer blend composition. A representative process for reducing the light oligomer content of an olefin feedstock can comprise (or consist essentially of, or consist of) contacting the olefin feedstock comprising C₆ to C₂₇ propylene oligomers with a catalyst composition comprising (i) an alpha-diimine nickel halide complex, (ii) a chemically-treated solid oxide, and (iii) optionally, a co-catalyst, to produce an oligomer composition. In this process, the number-average molecular weight (Mn) of the oligomer composition can be greater, by any amount disclosed herein, than that of the olefin feedstock.

Generally, the features of the processes (e.g., the olefin feedstock, the propylene oligomers, the catalyst composition, the alpha-diimine nickel halide complex, the chemically-treated solid oxide, the co-catalyst, the materials comprising and/or features of the composition, the oligomerization conditions under which the oligomer composition is formed, among others) are independently described herein, and these features can be combined in any combination to further describe the disclosed processes. Moreover, additional process steps can be performed before, during, and/or after any of the steps of any of the processes disclosed herein, unless stated otherwise.

The olefin feedstock comprising C₆ to C₂₇ propylene oligomers can come from many different sources and have a wide range of compositional attributes. As an example, the olefin feedstock can be derived from an oligomer product resulting from a propylene oligomerization process, the oligomer product containing predominantly C₃₀+ propylene oligomers, but having a significant portion of light oligomers: less than C₃₀, e.g., C₆ to C₂₇ propylene oligomers. This light oligomer fraction can be separated from the oligomer product using any suitable technique, such that a heavy propylene oligomer comprising C₃₀+ propylene oligomers and a light oligomer fraction comprising C₆ to C₂₇ propylene oligomers results. This latter fraction comprising C₆ to C₂₇ propylene oligomers can be the olefin feedstock in accordance with aspects of this invention.

In one aspect, at least about 50 wt. %, at least about 60 wt. %, at least about 70 wt. %, at least about 75 wt. %, or at least about 85 wt. %, of the olefin feedstock can be C₆ to C₂₇ propylene oligomers. In another aspect, the olefin feedstock can comprise at least about 90 wt. %, at least about 92 wt. %, at least about 95 wt. %, or at least about 97 wt. %, of C₆ to C₂₇ propylene oligomers. In yet another aspect, the C₆ to C₂₇ propylene oligomer content of the olefin feedstock can fall within a range from about 50 wt. % to 100 wt. %, from about 70 wt. % to 100 wt. %, from about 85 wt. % to 100 wt. %, from about 50 wt. % to about 99 wt. %, from about 70 wt. % to about 98 wt. %, or from about 85 wt. % to about 98 wt. %. In still another aspect, the C₆ to C₂₇ propylene oligomer content of the olefin feedstock can fall within a range from about 90 wt. % to 100 wt. %, from about 95 wt. % to 100 wt.

%, from about 90 wt. % to about 99 wt. %, from about 90 wt. % to about 98 wt. %, from about 92 wt. % to about 98 wt. %, or from about 95 wt. % to about 99 wt. %.

The oligomerization conditions under which the oligomer composition is produced can comprise any suitable oligomerization temperature. For example, the oligomerization temperature can be in a range from about 0° C. to about 150° C. In some aspects, the oligomerization temperature can be in a range from about 0° C. to about 100° C., from about 0° C. to about 60° C., or from about 0° C. to about 50° C., while in other aspects, the oligomerization temperature can be in a range from about 10° C. to about 100° C., from about 10° C. to about 80° C., from about 10° C. to about 50° C., or from about 10° C. to about 40° C. Yet, in further aspects, the oligomerization temperature can be in a range from about 15° C. to about 55° C., from about 15° C. to about 35° C., or from about 20° C. to about 40° C. Other appropriate oligomerization temperatures and temperature ranges are readily apparent from this disclosure.

The oligomerization conditions can comprise any suitable reaction pressure, whether ambient pressure, elevated pressure, or sub-atmospheric pressure.

In some aspects, the oligomer composition can be formed in the substantial absence of hydrogen. In these aspects, no hydrogen is added to the oligomerization reaction composition. As one of ordinary skill in the art would recognize, hydrogen can be generated in-situ by nickel complex catalyst compositions in various olefin oligomerization processes, and the amount generated can vary depending upon the specific catalyst composition and nickel halide complex employed, the type of oligomerization process used, the oligomerization reaction conditions utilized, and so forth.

In other aspects, it can be desirable to conduct the oligomerization process in the presence of a certain amount of added hydrogen, for instance, to reduce molecular weight, to reduce viscosity, etc. Accordingly, in these aspects, the oligomer composition can be formed in the presence of hydrogen, i.e., the olefin feedstock, the catalyst composition, and hydrogen can be contacted to form the oligomer composition. Any appropriate amount of hydrogen can be used.

Any suitable reactor or vessel within an oligomerization reaction system can be used to form the oligomer composition, non-limiting examples of which can include a fixed bed reactor, a stirred tank reactor, a plug flow reactor, and a loop slurry reactor, including more than one reactor in series or in parallel, and including any combination of reactor types and arrangements. In one aspect, the reaction system can comprise a single reactor (e.g., a single loop slurry rector or a single stirred tank reactor), while in another aspect, the reaction system can comprise two reactors in series (or parallel).

In the processes described herein, the catalyst composition can be deactivated after oligomerization. Deactivating the catalyst composition can comprise contacting the oligomer composition with a suitable catalyst composition deactivating agent, or subjecting the oligomer composition to suitable process steps to deactivate the catalyst composition, or a combination of both. The catalyst composition deactivating agent can comprise (or consist essentially of, or consist of) water, an alcohol compound, an amine compound, or any combination thereof; alternatively, water; alternatively, an alcohol compound; or alternatively, an amine compound. In an aspect, the alcohol compound can be a monoalcohol compound, a diol compound, a polyol compound, or any combination thereof. In some aspects, the alcohol compound can comprise, consist essentially of, or consist of, a C₁ to C₂₀ mono alcohol. In some aspects, the alcohol compound can comprise, consist essentially of, or consist of, methanol, ethanol, a propanol, a butanol, a pentanol, a hexanol, a heptanol, an octanol, a nonanol, a decanol, an undecanol, or mixtures thereof. In some aspects, the alcohol compound can comprise, consist essentially of, or consist of, methanol, ethanol, 1-propanol, 2-propanol, 1-butanol, 2-butanol, iso-butanol, sec-butanol, t-butanol, 1-hexanol, 2-hexanol, 3-hexanol, 1-heptanol, 2-heptanol, 3-heptanol, 4-heptanol, 1-octanol, 2-octanol, 3-octanol, 4-octanol, 2-ethyl-1-hexanol, 2-methyl-3-heptanol, 1-decanol, 2-decanol, 3-decanol, 4-decanol, 5-decanol, 1-undecanol, 2-undecanol, 7-methyl-2-decanol, a 1-docecanol, a 2-dodecanol, 2-ethyl-1-decanol, and mixtures thereof.

Additionally or alternatively, the catalyst composition can be deactivated by contact with an aqueous solution (e.g., an aqueous Group 1 metal hydroxide solution or an aqueous mineral acid solution). Such deactivation processes to deactivate the catalyst composition can also potentially remove a portion, or substantially all, of the metal catalyst composition components from the oligomer product.

In the processes described herein, the processes can further comprise a step of separating the oligomer composition from the catalyst composition or deactivated catalyst composition. Various suitable separations steps can be employed, as would be recognized by those of skill in the art. In an aspect, and not limited thereto, a filtration step can be used.

Beneficially, the number-average molecular weight (Mn) of the oligomer composition can be greater than that of the olefin feedstock, indicating a reduction in the amount of light oligomers. In aspects of this invention, the Mn of the oligomer composition can be greater than that of the olefin feedstock by at least about 5%, at least about 10%, at least about 15%, at least about 25%, at least about 35%, or at least about 40%, and in some instances, up to 50-100% greater. Additionally or alternatively, the weight-average molecular weight (Mw) of the oligomer composition can be greater than that of the olefin feedstock by at least about 5%, at least about 10%, at least about 15%, at least about 25%, at least about 35%, or at least about 40%, and in some instances, up to 50-100% greater.

Beneficially, the ratio of higher oligomer to lower oligomers (C₁₈+/C₁₅−) of the oligomer composition (on a molar basis) can be greater than that of the olefin feedstock, indicating a reduction in the amount of light oligomers. In aspects of this invention, the ratio of higher oligomer to lower oligomers (C₁₈+/C₁₅−) of the oligomer composition can be greater than that of the olefin feedstock by at least about 10%, at least about 25%, at least about 40%, at least about 75%, at least about 100%, at least about 500%, or at least about 1000%, and in some instances, up to 1500-2500% greater. As disclosed herein, C₁₈+/C₁₅− is the total C₁₈ to C₂₇ oligomers divided by the total C₆ to C₁₅ oligomers (on a molar basis).

In some aspects, the oligomer composition, in which the light oligomer fraction has been reduced, can be blended with a heavy propylene oligomer comprising C₃₀+ propylene oligomers. Thus, a process of this invention can comprise (a) contacting an olefin feedstock comprising C₆ to C₂₇ propylene oligomers with a catalyst composition comprising an alpha-diimine nickel halide complex, a chemically-treated solid oxide, and an optional a co-catalyst, to produce the oligomer composition, and (b) combining the oligomer composition with the heavy propylene oligomer comprising C₃₀+ propylene oligomers to form an oligomer blend composition. While not being limited thereto, the oligomer blend composition can contain greater than or equal to about 80 wt. % of the heavy propylene oligomer and less than or equal to about 20 wt. % of the oligomer composition. In further aspects, the oligomer blend composition can contain less than or equal to about 15 wt. %, or less than or equal to about 10 wt. %, of the oligomer composition. This invention also encompasses any oligomer compositions or oligomer blend compositions produced by any of the processes disclosed herein.

In an aspect, the processes described herein can further comprise a step of hydrogenating the oligomer composition (or the oligomer blend composition). Any suitable hydrogenation process and associated catalyst can be used, and such hydrogenation processes and catalysts (e.g., platinum, rhenium, palladium, nickel, etc.) are well known to those of skill in the art. Generally, the oligomer composition or the oligomer blend composition can be hydrogenated to provide a hydrogenated oligomer composition or hydrogenated oligomer blend composition having the desired degree of saturation (which can be quantified as a bromine number or bromine index). The oligomer composition or the oligomer blend composition can be hydrogenated to provide a hydrogenated oligomer composition or hydrogenated oligomer blend composition having any suitable bromine number or bromine index. In some aspects, the hydrogenated oligomer composition or hydrogenated oligomer blend composition can have a maximum bromine number of about 2, 1.8, 1.6, 1.4, 1.2, or 1 gram(s) of bromine per 100 grams of sample (g Br/100g). In other aspects, hydrogenated oligomer composition or hydrogenated oligomer blend composition described herein can have a maximum bromine index of about 1000, 800, 600, or 500 milligrams of bromine per 100 grams of sample (mg Br/100g). Generally, the bromine number can be determined by ASTM D1159-09, while the bromine index can be determined by ASTM D2710-09. This invention also encompasses any hydrogenated oligomer compositions or hydrogenated oligomer blend compositions produced by any of the processes disclosed herein.

Generally, the heavy propylene oligomer or the hydrogenated oligomer blend composition can have a viscosity index of at least 85 and a pour point in a range from −5 to −60° C. Moreover, the heavy propylene oligomer or the hydrogenated oligomer blend composition can be a liquid at standard temperature (25° C.) and pressure (1 atm). The heavy propylene oligomer or the hydrogenated oligomer blend composition also can have any of the properties provided below and in any combination.

In an aspect, the heavy propylene oligomer or the hydrogenated oligomer blend composition can have a pour point (ASTM D97-04) in a range from −5 to −50° C., from −5 to −45° C., from −10 to −40° C., from −10 to −35° C., from −15 to −60° C., from −15 to −50° C., or from −15 to −40° C.

In an aspect, the heavy propylene oligomer or the hydrogenated oligomer blend composition can have a viscosity index (ASTM D2270-10e1) in a range of from 85 to 200, from 85 to 175, from 85 to 140, from 85 to 130, from 88 to 150, from 88 to 135, from 90 to 140, or from 90 to 130.

In an aspect, the heavy propylene oligomer or the hydrogenated oligomer blend composition can have a kinematic viscosity at 100° C. (ASTM D7042-04) in a range from 6 to 200 cSt, from 8 to 150 cSt, from 10 to 150 cSt, from 10 to 100 cSt, from 12 to 150 cSt, from 12 to 100 cSt, from 12 to 80 cSt, from 12 to 60 cSt, or from 14 to 50 cSt.

In an aspect, the heavy propylene oligomer or the hydrogenated oligomer blend composition can have a kinematic viscosity at 40 ° C. (ASTM D7042-04) in a range from 25 to 8000 cSt, from 50 to 6000 cSt, from 75 to 6000 cSt, from 75 to 400 cSt, from 25 to 800 cSt, 100 to 6000 cSt, from 100 to 4000 cSt, from 150 to 6000, from 150 to 400 cSt, from 150 to 2000 cSt, from 175 to 2000 cSt, from 175 to 1500 cSt, from 200 to 2000 cSt, from 200 to 1500 cSt, or from 200 to 800 cSt.

In an aspect, the heavy propylene oligomer or the hydrogenated oligomer blend composition can have a flash point (ASTM D92-05) in a range from 140 to 300° C., from 140 to 260° C., from 140 to 220° C., from 140 to 190° C., from 160 to 240° C., or from 160 to 200° C.

This invention also contemplates and encompasses any compositions (e.g., lubricant compositions or lubricant formulations) or base oils that comprise the oligomer compositions (or hydrogenated oligomer compositions) or oligomer blend compositions (or hydrogenated oligomer blend compositions) disclosed herein. Such lubricant compositions or formulations can include one or more suitable additives, such as viscosity index improvers/viscosity modifiers/viscosity improvers, dispersants (metallic and/or non-metallic), detergents (metallic and/or non-metallic), friction modifiers, traction improving additives, demulsifiers, defoamants, antioxidants, anti-wear additives (metallic and non-metallic, phosphorus-containing and non-phosphorus, sulfur-containing and non-sulfur types), extreme-pressure additives (metallic and non-metallic, phosphorus-containing and non-phosphorus, sulfur-containing and non-sulfur types), anti-rust additives, corrosion inhibitors, metal deactivators, anti-seizure agents, pour point depressants, wax modifiers, seal compatibility agents, friction modifiers, lubricity agents, anti-staining agents, chromophores (dyes), haze inhibitors, and the like. Additional information on additives used in lubricant formulations can be found in “Fuels and Lubricants Handbook: Technology, Properties, Performance, and Testing” edited by George E. Totten, Steven R. Westbrook, Rajesh J. Shah, ASTM (2003), ISBN 0-8031-2096-6; Chapter 9 Additives and Additive Chemistry, pp. 199-248, “Lubricants and Related Products,” Klamann, Verlag Chemie, Deerfield Beach, FL, ISBN 0-89573-177-0; “Lubricant Additives” by M. W. Ranney, published by Noyes Data Corporation of Parkridge, N.J. (1973); “Lubricants and Lubrications,” T. Mang and W. Dresel, eds., Wiley-VCH GmbH, Weinheim (2001); and “Lubricant Additives”, C. V. Smallheer and R. K. Smith, published by the Lezius-Hiles Co. of Cleveland, Ohio (1967).

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

Relative oligomer product distributions (C₆, C₉, C₁₂, C₁₅, C₁₈, C₂₁, C₂₄, C₂₇) were determined by integration of the GC-FID spectra and are reported in mole percentages. Spectral assignment was made using GC-MS. Any C₃₀+ propylene oligomers were not included. Mn is the number-average molecular weight, and Mw is the weight-average molecular weight.

Gas chromatography was performed using a Varian 3800 GC analyzer equipped with two separate all-purpose capillary columns (Agilent J&W VF-5ms, 30 m×0.25 mm×0.25 μm) and a flame ionization (FI) detector. 1 μL sample aliquots were injected into a GC port held at 250° C. using a split ratio of 20:1. The carrier gas used was ultra-high purity helium and was electronically controlled throughout the run to a constant flow rate of 1.0 mL/min. Initial column temperature was held at 70° C. for 2 min, ramped at 20° C./min to 250° C., and then held at 250° C. for 19 min. The FI detector on the GC was maintained at 300° C. Spectral assignment was made via mass correlation using a Varian 320 MS mass spectrometer connected to the GC unit using electron ionization at 70 eV. The nominal mass range scanned was 50-900 m/z using a scan time of 0.5 s. Nominal detector voltage used was 1200 V.

Fluorided silica-coated alumina (FSCA) was prepared as follows. Alumina A from W.R. Grace having a surface area of 300 m²/g, a pore volume of 1.2 mL/g, and an average particle size of 100 microns, was first calcined in dry air for 6 hours at 600° C., then cooled to ambient temperature, followed by contacting with tetraethylorthosilicate in isopropanol to equal 25 wt. % SiO₂. After drying, the silica-coated alumina was calcined at 600° C. for 3 hours. Fluorided silica-coated alumina (7 wt. % F) was prepared by impregnating the calcined silica-coated alumina with an ammonium bifluoride solution in methanol, drying, and then calcining for 3 hours at 600° C. in dry air. Afterward, the fluorided silica-coated alumina (FSCA) was collected and stored under dry nitrogen, and was used without exposure to the atmosphere.

Sulfated alumina (SA) was prepared as follows. Alumina A was first calcined in dry air for 5 hours at 600° C., then cooled to ambient temperature. The calcined alumina was impregnated with a solution of sulfuric acid in methanol, followed by drying, and calcining for 3 hours at 600° C. in dry air. Afterward, the sulfated alumina (SA) was collected and stored under dry nitrogen, and was used without exposure to the atmosphere.

Fluorided silica-alumina (F-SiAl) was prepared as follows. A silica-alumina was obtained from W.R. Grace Company containing 13% alumina by weight and having a surface area of 400 m²/g, a pore volume of 1.2 mL/g, and an average particle size of 70 microns. Approximately 100 grams of this material were impregnated with a solution containing about 200 mL of water and about 10 grams of ammonium hydrogen fluoride, resulting in a damp powder having the consistency of wet sand. After drying, the fluorided silica-alumina was calcined for 3 hours at 450 ° C. in dry air. Afterward, the fluorided silica-alumina (F-SiAl) was collected and stored under dry nitrogen, and was used without exposure to the atmosphere.

In the examples that follow, Met-1 is the metallocene compound shown below, Ni-1 is the alpha-diimine nickel bromide complex shown below, and Pd-1 is the palladium analogue of the alpha-diimine nickel bromide complex (Ni-1):

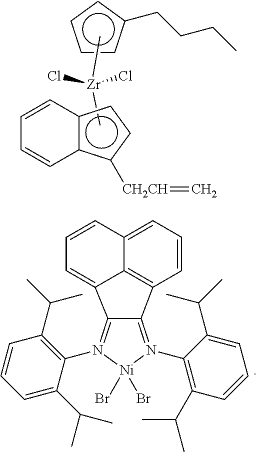

### Examples A-D

Propylene oligomers were produced using the following equipment and procedure. In a glove box, a syringe was charged sequentially with fluorided silica-coated alumina (FSCA), hexanes, triisobutylaluminum (TIBA, 1 M solution in heptanes), and a 2 mg/mL toluene solution containing the desired catalyst. The slurry was charged to an autoclave, and the autoclave was then charged with liquid propylene at 250-300 psig. The desired amount of hydrogen was charged and the autoclave was then heated to the desired temperature and the reaction allowed to proceed, with stirring, for one hour. The reactor was then cooled to 40° C. The reactor was vented to a flare line to allow the unreacted propylene to vent from the reactor. The liquid product was collected and filtered to remove the chemically-treated solid oxide. The filtrate was then distilled to a pot temperature of 130° C. at less than 0.5 torr. The distillate (generally, C₆-C₂₇ propylene oligomers; some C₃₀) was condensed using a chilled receiver and then used without further purification.

Table I summarizes the propylene oligomerizations of Examples A-D. In Table I, autoclave size (gallons) is the reactor size that was used for each experiment, catalyst (mg) is the weight of the metallocene and/or nickel halide compound used, FSCA (mg) is the weight of the fluorided silica-coated alumina used, TIBA (mL) is the amount of a 1M triisobutylaluminum solution of co-catalyst used, propylene (L) is the amount of propylene reactant charged to the autoclave reactor, T (° C.) is the oligomerization reaction temperature used, H₂ (mg) is the amount of hydrogen used, crude yield (g) is the weight of the crude oligomer product produced prior to distillation, and distillate yield (wt. %) is the relative amount of C₆-C₂₇ propylene oligomers recovered from the crude oligomer product.

### Examples 1-15

Table II summarizes the distillate oligomerization experiments of Examples 1-15 (using a nickel or palladium complex as the catalyst), in which the olefin feedstock that was oligomerized was the distillate product of Examples A-D. For Examples 1-4, a 20 mL vial was charged with the indicated distillate (olefin feedstock), 1M TIBA solution, chemically treated solid oxide (CTSO), and catalyst. The resulting slurry was stirred for 18 hr at ambient temperature (20-25° C.). Then, 5 mL of water was added and the mixture was filtered. The filtrate was phase separated and the organic layer was dried on MgSO₄. The slurry was filtered and the filtrate was sampled for analytical testing.

For Examples 5-8, a 20 mL vial was charged with the indicated distillate (olefin feedstock), TIBA, CTSO, and catalyst. The resulting slurry was heated to 70° C. and stirred for 2.5 hr. Then, 5 mL of water was added and the mixture was filtered. The filtrate was phase separated and the organic layer was dried on MgSO₄. The slurry was filtered and the filtrate was sampled for analytical testing.

For Examples 9-10, a 20 mL vial was charged with the indicated distillate (olefin feedstock), TIBA, CTSO, and catalyst. The resulting slurry was heated to 70° C. and stirred for 6 hr. Then, 5 mL of water was added and the mixture was filtered. The filtrate was phase separated and the organic layer was dried on MgSO₄. The slurry was filtered and the filtrate was sampled for analytical testing.

For Examples 11-12, a 20 mL vial was charged with indicated distillate (olefin feedstock), TIBA, CTSO, and catalyst. The resulting slurry was stirred for 56 hr at ambient temperature (20-25° C.). Then, 5 mL of water was added and the mixture was filtered. The filtrate was phase separated and the organic layer was dried on MgSO₄. The slurry was filtered and the filtrate was sampled for analytical testing.

For Examples 13-15, a 20 mL vial was charged with indicated distillate (olefin feedstock), TIBA, CTSO, and catalyst. The resulting slurry was stirred for 15 hr at ambient temperature (20-25° C.). Then, 5 mL of water was added and the mixture was filtered. The filtrate was phase separated and the organic layer was dried on MgSO₄. The slurry was filtered and the filtrate was sampled for analytical testing.

For the distillates of Examples A-D (olefin feedstocks) and the oligomer compositions of Examples 1-11 and 13-15, Table III summarizes the propylene oligomer distribution (C₆, C₉, C₁₂, C₁₅, C₁₈, C₂₁, C₂₄, and C₂₇) in mole percentages, number-average molecular weight (Mn) in g/mol, weight-average molecular weight (Mw) in g/mol, total C₁₈-C₂₇, total C₆-C₁₅, and the ratio of C₁₈+/C₁₅− (i.e., total of C₁₈-C₂₇ divided by the total of C₆-C₁₅). For each carbon number, some material may be saturated (two additional carbons), but this was expected to be a minor amount, so the following molecular weights for each carbon number were used (g/mol): C₆=84, C₉=126, C₁₂=168, C₁₅=210, C₁₈=252, C₂₁=294, C₂₄=336, and C₂₇=378.

As shown by comparing Examples 1-2 to Example A in Table III, and unexpectedly, the catalyst systems disclosed herein (containing an alpha-diimine nickel halide complex) were able to significantly increase the molecular weight of a light oligomer fraction (less than C₃₀). This result was also confirmed by comparing Examples 3-4 to Example B. Examples 1-4 resulted in ˜10-40% increases in the Mn of the oligomer compositions as compared to the olefin feedstocks (Examples A-B). Similarly, the Mw increased by ˜15-45%.

As shown in Table III, the ratios of C₁₈+/C₁₅− also increased significantly. Examples 1-4 resulted in ˜80-1800% increases in the the ratio of C₁₈+/C₁₅− of the oligomer compositions as compared to that of the olefin feedstocks (Examples A-B).

Examples 5-10 were conducted at an oligomerization temperature of 70° C., and the results in Table III indicate that a lower oligomerization temperature, such as 20-25° C., may be more effective. The remaining examples in Table III showed negligible to small reductions in the light oligomer fraction of olefin feedstock D, as reflected by the Mn, Mw, and ratio of C₁₈+/C₁₅−.

TABLE I
Examples A-D

| | Autoclave | | | | | | | Crude | Distillate |
|---|---|---|---|---|---|---|---|---|---|
| | size | Catalyst | FSCA | TIBA | Propylene | T | H₂ | Yield | Yield |
| Example | (gallons) | (mg) | (mg) | (mL) | (L) | (° C.) | (mg) | (g) | (wt. %) |
| A | 5.0 | 30 Met-1 | 4500 | 2.0 | 11.4 | 77 | 0 | 1075 | N/A |
| B | 1.0 | 2 Met-1 | 300 | 0.5 | 2.4 | 77 | 101 | 126 | N/A |
| C | 0.26 | 2 Met-1 + 2 Ni-1 | 500 | 0.6 | 0.6 | 77 | 8 | 37.4 | 13.6 |
| D | 1.0 | 10 Met-1 | 750 | 0.5 | 2.4 | 75 | 101 | 236.6 | 15.7 |

TABLE II
Examples 1-15

| | Olefin | | | Catalyst | Feedstock | | CTSO | TIBA | Temperature | Time |
|---|---|---|---|---|---|---|---|---|---|---|
| Example | Feedstock | Catalyst | CTSO | (mg) | Amount | | (mg) | (mL) | (° C.) | (hr) |
| 1 | A | Ni-1 | FSCA | 3.1 | 3.0 | mL | 150 | 0.5 | Ambient | 18 |
| 2 | A | Ni-1 | SA | 2.9 | 3.0 | mL | 150 | 0.5 | Ambient | 18 |
| 3 | B | Ni-1 | FSCA | 3.9 | 3.0 | mL | 150 | 0.5 | Ambient | 18 |
| 4 | B | Ni-1 | SA | 3.1 | 3.0 | mL | 150 | 0.5 | Ambient | 18 |
| 5 | C | Ni-1 | FSCA | 1 | 1.0 | g | 100 | 0.25 | 70 | 2.5 |
| 6 | C | Ni-1 | SA | 1 | 1.0 | g | 100 | 0.25 | 70 | 2.5 |
| 7 | C | Pd-1 | FSCA | 1 | 1.0 | g | 100 | 0.25 | 70 | 2.5 |
| 8 | C | Pd-1 | SA | 1 | 1.0 | g | 100 | 0.25 | 70 | 2.5 |
| 9 | D | Ni-1 | FSCA | 5 | 4.1 | g | 250 | 0.9 | 70 | 6 |
| 10 | D | Ni-1 | FSCA | 5.2 | 4.2 | g | 250 | 0.9 | 70 | 6 |
| 11 | D | Ni-1 | FSCA | 5 | 4.1 | g | 250 | 0.9 | Ambient | 56 |
| 12 | D | Ni-1 | FSCA | 5 | 4.0 | g | 250 | 0.9 | Ambient | 56 |
| 13 | D | Ni-1 | FSCA | 5 | 1.5 | g | 100 | 0.25 | Ambient | 15 |
| 14 | D | Ni-1 | F—SiAl | 5 | 1.5 | g | 100 | 0.25 | Ambient | 15 |
| 15 | D | Ni-1 | SA | 5 | 1.5 | g | 100 | 0.25 | Ambient | 15 |

TABLE III
Product Distributions and Molecular Weights
for Examples A-D and Examples 1-11 and 13-15

| Example | C₆ | C₉ | C₁₂ | C₁₅ | C₁₈ | C₂₁ | C₂₄ | C₂₇ |
|---|---|---|---|---|---|---|---|---|
| A | 3.69 | 16.46 | 26.84 | 23.79 | 19.40 | 6.94 | 2.48 | 0.40 |
| 1 | 3.51 | 11.25 | 21.40 | 20.45 | 17.82 | 13.13 | 8.08 | 4.36 |
| 2 | 9.40 | 10.29 | 17.47 | 18.19 | 16.83 | 13.37 | 9.13 | 5.33 |
| B | 7.35 | 25.40 | 47.03 | 15.73 | 3.74 | 0.71 | 0.04 | 0.00 |
| 3 | 3.96 | 7.82 | 25.06 | 16.38 | 17.31 | 17.04 | 10.68 | 1.75 |
| 4 | 12.71 | 24.47 | 11.32 | 19.90 | 6.08 | 5.13 | 16.09 | 4.30 |
| C | 4.11 | 20.04 | 31.98 | 25.40 | 12.49 | 4.81 | 1.08 | 0.08 |
| 5 | 3.80 | 18.20 | 33.40 | 25.72 | 15.00 | 3.47 | 0.34 | 0.00 |
| 6 | 4.68 | 25.39 | 32.33 | 25.71 | 9.50 | 2.29 | 0.10 | 0.00 |
| 7 | 6.89 | 20.41 | 37.14 | 24.21 | 9.28 | 2.01 | 0.05 | 0.00 |
| 8 | 5.54 | 19.63 | 34.35 | 27.17 | 10.45 | 2.59 | 0.26 | 0.00 |
| D | 12.01 | 26.46 | 35.16 | 25.98 | 0.40 | 0.00 | 0.00 | 0.00 |
| 9 | 11.96 | 23.22 | 34.74 | 29.49 | 0.49 | 0.09 | 0.00 | 0.00 |
| 10 | 11.59 | 23.92 | 35.19 | 28.83 | 0.42 | 0.06 | 0.00 | 0.00 |
| 11 | 11.00 | 22.65 | 35.09 | 30.63 | 0.70 | 0.05 | 0.00 | 0.00 |
| 13 | 10.52 | 26.19 | 34.64 | 27.76 | 0.71 | 0.10 | 0.08 | 0.00 |
| 14 | 11.96 | 24.93 | 35.12 | 27.43 | 0.55 | 0.00 | 0.00 | 0.00 |
| 15 | 7.61 | 22.34 | 36.14 | 31.85 | 1.45 | 0.47 | 0.15 | 0.00 |

| Example | Mn | Mw | Total C₁₈-C₂₇ | Total C₆-C₁₅ | C₁₈+/C₁₅− |
|---|---|---|---|---|---|
| A | 198.0 | 215.3 | 29.22 | 70.78 | 41.3 |
| 1 | 223.2 | 247.0 | 43.39 | 56.61 | 76.7 |
| 2 | 220.9 | 250.8 | 44.65 | 55.35 | 80.7 |
| B | 161.9 | 171.7 | 4.49 | 95.51 | 4.7 |
| 3 | 225.9 | 248.3 | 46.78 | 53.22 | 87.9 |
| 4 | 203.0 | 243.1 | 31.60 | 68.40 | 46.2 |
| C | 185.3 | 200.3 | 18.46 | 81.54 | 22.6 |
| 5 | 185.4 | 198.7 | 18.81 | 81.11 | 23.2 |
| 6 | 175.2 | 188.0 | 11.89 | 88.11 | 13.5 |
| 7 | 174.2 | 187.0 | 11.34 | 88.66 | 12.8 |
| 8 | 179.0 | 191.9 | 13.31 | 86.69 | 15.3 |
| D | 158.1 | 168.8 | 0.40 | 99.61 | 0.4 |
| 9 | 161.1 | 172.1 | 0.58 | 99.41 | 0.6 |
| 10 | 160.8 | 171.6 | 0.48 | 99.53 | 0.5 |
| 11 | 163.0 | 173.5 | 0.75 | 99.37 | 0.8 |
| 13 | 160.7 | 171.4 | 0.89 | 99.11 | 0.9 |
| 14 | 159.4 | 170.3 | 0.55 | 99.44 | 0.6 |
| 15 | 167.7 | 177.9 | 2.07 | 97.94 | 2.1 |

The invention is described above with reference to numerous aspects and specific examples. Many variations will suggest themselves to those skilled in the art in light of the above detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A process comprising:

contacting an olefin feedstock comprising C₆ to C₂₇ propylene oligomers with a catalyst composition comprising (i) an alpha-diimine nickel halide complex, (ii) a chemically-treated solid oxide, and (iii) optionally, a co-catalyst, to produce an oligomer composition, wherein a number-average molecular weight (Mn) of the oligomer composition is greater than that of the olefin feedstock.

Aspect 2. The process defined in aspect 1, wherein the number-average molecular weight (Mn) of the oligomer composition is greater than that of the olefin feedstock by any amount disclosed herein, e.g., at least about 5%, at least about 10%, at least about 25%, at least about 40%, etc.

Aspect 3. The process defined in aspect 1 or 2, wherein a ratio of higher oligomers to lower oligomers (C₁₈+/C₁₅−) of the oligomer composition is greater than that of the olefin feedstock by any amount disclosed herein, e.g., at least about 25%, at least about 40%, at least about 100%, at least about 500%, at least about 1000%, etc.

Aspect 4. The process defined in any one of the preceding aspects, wherein the chemically-treated solid oxide comprises a solid oxide treated with an electron-withdrawing anion, e.g., any solid oxide and any electron-withdrawing anion disclosed herein.

Aspect 5. The process defined in aspect 4, wherein (a) the solid oxide comprises silica, alumina, silica-alumina, silica-coated alumina, aluminum phosphate, aluminophosphate, heteropolytungstate, titania, zirconia, magnesia, boria, zinc oxide, a mixed oxide thereof, or any mixture thereof, and (b) the electron-withdrawing anion comprises sulfate, bisulfate, fluoride, chloride, bromide, iodide, fluorosulfate, fluoroborate, phosphate, acetate, fluorophosphate, trifluoroacetate, triflate, fluorozirconate, fluorotitanate, phospho-tungstate, or any combination thereof.

Aspect 6. The process defined in aspect 4 or 5, wherein the solid oxide comprises silica, alumina, silica-alumina, silica-coated alumina, or a mixture thereof.

Aspect 7. The process defined in aspect 4 or 5, wherein the solid oxide comprises silica-coated alumina.

Aspect 8. The process defined in any one of aspects 4-7, wherein the electron-withdrawing anion comprises sulfate, fluoride, chloride, or any combination thereof.

Aspect 9. The process defined in any one of aspects 4-7, wherein the electron-withdrawing anion comprises sulfate.

Aspect 10. The process defined in any one of aspects 4-7, wherein the electron-withdrawing anion comprises fluoride, chloride, or both.

Aspect 11. The process defined in any one of aspects 1-4, wherein the chemically-treated solid oxide comprises fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, or any combination thereof.

Aspect 12. The process defined in any one of aspects 1-4, wherein the chemically-treated solid oxide comprises a fluorided solid oxide, a sulfated solid oxide, or a combination thereof.

Aspect 13. The process defined in any one of aspects 1-4, wherein the chemically-treated solid oxide comprises fluorided alumina, sulfated alumina, fluorided silica-alumina, sulfated silica-alumina, fluorided silica-coated alumina, fluorided-chlorided silica-coated alumina, sulfated silica-coated alumina, or any combination thereof.

Aspect 14. The process defined in any one of aspects 1-4, wherein the chemically-treated solid oxide comprises fluorided silica-coated alumina.

Aspect 15. The process defined in any one of aspects 1-4, wherein the chemically-treated solid oxide comprises sulfated alumina.

Aspect 16. The process defined in any one of the preceding aspects, wherein the catalyst composition comprises a co-catalyst, e.g., any co-catalyst disclosed herein.

Aspect 17. The process defined in any one of the preceding aspects, wherein the co-catalyst comprises an organoaluminum compound, an organozinc compound, an organomagnesium compound, an organolithium compound, or any combination thereof.

Aspect 18. The process defined in any one of the preceding aspects, wherein the co-catalyst comprises an organoaluminum compound.

Aspect 19. The process defined in aspect 18, wherein the organoaluminum compound comprises any organoaluminum compound disclosed herein, e.g., trimethylaluminum, triethylaluminum, triisobutylaluminum, etc., or combinations thereof.

Aspect 20. The process defined in any one of aspects 1-19, wherein the catalyst composition is substantially free of aluminoxane compounds, organoboron or organoborate compounds, ionizing ionic compounds, or combinations thereof.

Aspect 21. The process defined in any one of aspects 1-16, wherein the co-catalyst comprises an aluminoxane compound, an organoboron or organoborate compound, an ionizing ionic compound, an organoaluminum compound, an organozinc compound, an organomagnesium compound, an organolithium compound, or any combination thereof.

Aspect 22. The process defined in any one of aspects 1-21, wherein the alpha-diimine nickel halide complex comprises an alpha-diimine nickel chloride complex.

Aspect 23. The process defined in any one of aspects 1-21, wherein the alpha-diimine nickel halide complex comprises an alpha-diimine nickel bromide complex.

Aspect 24. The process defined in any one of aspects 1-21, wherein the alpha-diimine nickel

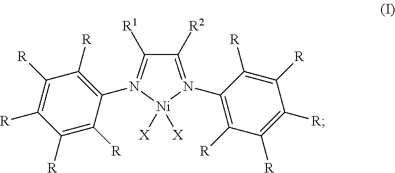

halide complex has formula (I):

wherein each R independently is any suitable substituent or any substituent disclosed herein, R¹ and R² independently are any suitable substituent or any substituent disclosed herein, or R¹ and R² are linked to form any suitable ring system or any ring system disclosed herein, and each X independently is any suitable halide or any halide disclosed herein.

Aspect 25. The process defined in aspect 24, wherein each X is Cl.

Aspect 26. The process defined in aspect 24, wherein each X is Br.

Aspect 27. The process defined in any one of aspects 24-26, wherein each R independently is H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group.

Aspect 28. The process defined in any one of aspects 24-26, wherein each R independently is H or a C₁ to C₁₈ hydrocarbyl group.

Aspect 29. The process defined in any one of aspects 24-26, wherein each R independently is H or a C₁ to C₈ alkyl group.

Aspect 30. The process defined in any one of aspects 24-29, wherein R¹ and R² independently are H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group.

Aspect 31. The process defined in any one of aspects 24-29, wherein R¹ and R² independently are H or a C₁ to C₁₈ hydrocarbyl group.

Aspect 32. The process defined in any one of aspects 24-29, wherein R¹ and R² independently are H or a C₁ to C₈ alkyl group.

Aspect 33. The process defined in any one of aspects 24-29, wherein R¹ and R² are linked to form a cycloalkyl ring or ring system.

Aspect 34. The process defined in any one of aspects 24-29, wherein R¹ and R² are linked to form an aromatic ring or ring system.

Aspect 35. The process defined in any one of the preceding aspects, wherein the catalyst composition is produced by a process comprising contacting, in any order, the alpha-diimine nickel halide complex, the chemically-treated solid oxide, and the co-catalyst.

Aspect 36. The process defined in any one of the preceding aspects, wherein a weight ratio of the chemically-treated solid oxide to the alpha-diimine nickel halide complex is in any range of weight ratios disclosed herein, e.g., from about 5:1 to about 1000:1, from about 5:1 to about 500:1, from about 10:1 to about 800:1, from about 10:1 to about 300:1, from about 10:1 to about 200:1, from about 10:1 to about 150:1, from about 20:1 to about 100:1, etc.

Aspect 37. The process defined in any one of the preceding aspects, wherein a weight ratio of the co-catalyst to the alpha-diimine nickel halide complex is in any range of weight ratios disclosed herein, e.g., from about 1:10 to about 100:1, from about 1:1 to about 100:1, from about 5:1 to about 75:1, from about 10:1 to about 50:1, etc.

Aspect 38. The process defined in any one of the preceding aspects, wherein a weight ratio of the olefin feedstock (and/or the C₆ to C₂₇ propylene oligomers) to the alpha-diimine nickel halide complex is in any range of weight ratios disclosed herein, e.g., from about 50:1 to about 8000:1, from about 50:1 to about 5000:1, from about 100:1 to about 5000:1, from about 100:1 to about 2500:1, from about 200:1 to about 2000:1, from about 250:1 to about 1500:1, etc.

Aspect 39. The process defined in any one of the preceding aspects, wherein the oligomer composition is produced under oligomerization conditions comprising any suitable oligomerization temperature or an oligomerization temperature in any oligomerization temperature range disclosed herein, e.g., from about 0° C. to about 150° C., from about 10° C. to about 100° C., from about 10° C. to about 80° C., from about 0° C. to about 50° C., from about 10° C. to about 50° C., from about 10° C. to about 40° C., from about 15° C. to about 35° C., from about 20° C. to about 40° C., etc.

Aspect 40. The process defined in any one of the preceding aspects, wherein the olefin feedstock comprises any suitable amount of the C₆ to C₂₇ propylene oligomers or an amount of the C₆ to C₂₇ propylene oligomers in any range disclosed herein, e.g., at least about 50 wt. %, at least about 75 wt. %, at least about 90 wt. %, at least about 95 wt. %, from about 70 wt. % to 100 wt. %, from about 90 wt. % to 100 wt. %, from about 90 wt. % to about 99 wt. %, etc.

Aspect 41. The process defined in any one of the preceding aspects, wherein the oligomer composition is produced in the substantial absence of hydrogen (e.g., no added hydrogen).

Aspect 42. The process defined in any one of the preceding aspects, wherein the oligomer composition is produced in a reaction system comprising a fixed bed reactor, a stirred tank reactor, a plug flow reactor, a loop slurry reactor, or a combination thereof.

Aspect 43. The process defined in any one of the preceding aspects, wherein the process further comprises a step of deactivating the catalyst composition using any suitable technique or any technique disclosed herein.

Aspect 44. The process defined in any one of the preceding aspects, wherein the process further comprises a step of separating the oligomer composition from the catalyst composition or deactivated catalyst composition using any suitable technique or any technique disclosed herein, e.g., filtration, etc.

Aspect 45. The process defined in any one of the preceding aspects, wherein the process further comprises a step of combining the oligomer composition with a heavy propylene oligomer comprising C₃₀+ propylene oligomers to form an oligomer blend composition.

Aspect 46. The process defined in any one of the preceding aspects, wherein the process further comprises a step of hydrogenating the oligomer composition or the oligomer blend composition using any suitable technique or any technique disclosed herein.

Aspect 47. An oligomer composition (or hydrogenated oligomer composition) or oligomer blend composition (or hydrogenated oligomer blend composition) produced by the process defined in any one of the preceding aspects.

Aspect 48. A lubricant composition comprising the oligomer composition (or hydrogenated oligomer composition) or oligomer blend composition (or hydrogenated oligomer blend composition) defined in aspect 47.

Aspect 49. The lubricant composition defined in aspect 48, further comprising any suitable additive or any additive disclosed herein, as well as combinations thereof.

1. A process comprising:
contacting an olefin feedstock comprising C₆ to C₂₇ propylene oligomers with a catalyst composition comprising (i) an alpha-diimine nickel halide complex, (ii) a chemically-treated solid oxide, and (iii) optionally, a co-catalyst, to produce an oligomer composition, wherein a number-average molecular weight (Mn) of the oligomer composition is greater than that of the olefin feedstock.

2. The process of claim 1, wherein the Mn of the oligomer composition is greater than that of the olefin feedstock by from about 5% to about 100%.

3. The process of claim 1, wherein a ratio of a total of C₁₈ to C₂₇ oligomers to a total of C₆ to C₁₅ oligomers of the oligomer composition is greater than that of the olefin feedstock by from about 25% to about 2500%.

4. The process of claim 1, wherein:
the catalyst composition comprises a co-catalyst; and
the chemically-treated solid oxide comprises a fluorided solid oxide and/or a sulfated solid oxide.

5. The process of claim 4, wherein the co-catalyst comprises an organoaluminum compound.

6. The process of claim 1, wherein the chemically-treated solid oxide comprises fluorided alumina, sulfated alumina, fluorided silica-alumina, sulfated silica-alumina, fluorided silica-coated alumina, fluorided-chlorided silica-coated alumina, sulfated silica-coated alumina, or any combination thereof.

7. The process of claim 1, wherein the alpha-diimine nickel halide complex comprises an alpha-diimine nickel chloride complex.

8. The process of claim 1, wherein the alpha-diimine nickel halide complex comprises an alpha-diimine nickel bromide complex.

9. The process of claim 1, wherein the alpha-diimine nickel halide complex has formula (I): 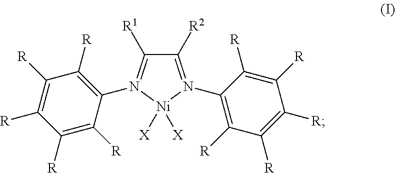
wherein:
each R independently is H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group;
R¹ and R² independently are H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group, or R¹ and R² are linked to form a ring or ring system; and
each X independently is a halide.

10. The process of claim 9, wherein:
each R independently is H or a C₁ to C₈ alkyl group;
R¹ and R² independently are H or a C₁ to C₁₈ hydrocarbyl group; and
each X independently is Cl or Br.

11. The process of claim 9, wherein:
each R independently is H or a C₁ to C₈ alkyl group;
R¹ and R² are linked to form a cycloalkyl ring or ring system or an aromatic ring or ring system; and
each X independently is Cl or Br.

12. The process of claim 1, wherein a weight ratio of the chemically-treated solid oxide to the alpha-diimine nickel halide complex is in a range from about 10:1 to about 200:1.

13. The process of claim 1, wherein:
the catalyst composition comprises the co-catalyst and
a weight ratio of the co-catalyst to the alpha-diimine nickel halide complex is in a range from about 5:1 to about 75:1.

14. The process of claim 1, wherein:
a weight ratio of the C₆ to C₂₇ propylene oligomers to the alpha-diimine nickel halide complex is in a range from about 100:1 to about 2500:1.

15. The process of claim 1, wherein:
the oligomer composition is produced at an oligomerization temperature in a range from about 10° C. to about 50° C.; and
the olefin feedstock contains at least about 50 wt. % of the C₆ to C₂₇ propylene oligomers.

16. The process of claim 1, wherein:
the oligomer composition is produced at an oligomerization temperature in a range from about 15° C. to about 35° C.; and
the olefin feedstock contains at least about 95 wt. % of the C₆ to C₂₇ propylene oligomers.

17. The process of claim 1, wherein the process further comprises a step of combining the oligomer composition with a heavy propylene oligomer comprising C₃₀+ propylene oligomers to form an oligomer blend composition.

18. The process of claim 17, wherein the oligomer blend composition comprises less than or equal to about 20 wt. % of the oligomer composition.

19. The process of claim 17, wherein the process further comprises a step of hydrogenating the oligomer blend composition to form a hydrogenated oligomer blend composition.

20. The process of claim 19, wherein the hydrogenated oligomer blend composition is characterized by:
a flash point in a range from about 140 to about 260° C.;
a viscosity index in a range from about 85 to about 130; and
a pour point in a range from about −10 to about −40° C.
